(19) United States
(12) Patent Application Publication
(10) Pub. No.: US 2022/0221458 A1
(43) Pub. Date: Jul. 14, 2022

(54) NOROVIRUS-BINDING PEPTIDE

(71) Applicant: Kao Corporation, Chuo-ku, Tokyo (JP)

(72) Inventors: Yoshitaka ITO, Sakura-shi, Tochigi (JP); Takuya MORIMOTO, Ota-ku, Tokyo (JP); Shigefumi KUMACHI, Saitama-shi, Saitama (JP); Naoto NEMOTO, Saitama-shi, Saitama (JP)

(73) Assignee: Kao Corporation, Chuo-ku, Tokyo (JP)

(21) Appl. No.: 17/613,287

(22) PCT Filed: May 28, 2020
(86) PCT No.: PCT/JP2020/021237

§ 371 Date: Nov. 22, 2021

(30) Foreign Application Priority Data
May 29, 2019 (JP) 2019-100756

(51) Int. Cl. G01N 33/569; C07K 7/06

(52) U.S. Cl. CPC G01N 33/56983; G01N 2333/08; C07K 7/06

(57) ABSTRACT

A peptide that specifically binds to norovirus, which is useful for detection and infection control of norovirus is provided. A norovirus-binding peptide consisting of an amino acid sequence selected from the group consisting of SEQ ID NOs: 1 to 269.

### FIELD OF THE INVENTION

The present invention relates to a norovirus-binding peptide having affinity to norovirus.

### BACKGROUND OF THE INVENTION

Norovirus is a virus that has been called SRSV (Small Round Structured Virus) and also called NLV (Norwalk-like virus), and is classified into five categories from GI (genogroup I) to GV (genogroup V) based on the genotypes, among which GI, GII, and GIV infect humans. Norovirus is a virus that propagates in human intestinal cells and causes food poisoning with symptoms such as diarrhea, vomiting, abdominal pain, nausea, and fever. The main source of infection is food, and raw oysters are often a problem. In addition, in recent years, human to human transmission through excrement etc. of a virus carrier is also increasing.

Currently, as the detection of norovirus, in addition to observation with an electron microscope, there are a method using an antibody and a method of measuring the amount of an amplification product of norovirus RNA. Furthermore, recently, a polypeptide consisting of 18 amino acids that has affinity to norovirus and is useful for detection of norovirus has also been found, but it has been reported that the bonding strength is low compared to previously reported norovirus antibodies (Non Patent Literature 1).

However, a method for detecting RNA requires reverse transcription and an amplification step, and the operation is complicated and takes time and cost. Antibodies also have problems: the specificity is low in some cases; and since animals or culture cells are used for producing and manufacturing antibodies, the quality is unstable and the cost is high.

Accordingly, there is a demand for developing a more effective and simpler norovirus-specific detection method and a prophylactic and therapeutic method for norovirus infection.

### Non Patent Literature 1

Hye Jin Hwang, et al., Biosensors and Bioelectronics, 2017, 87, 164-170

### SUMMARY OF THE INVENTION

The present invention relates to the following 1) to 4):

1) a norovirus-binding peptide consisting of an amino acid sequence selected from the group consisting of SEQ ID NOs: 1 to 269;

2) a norovirus-binding peptide consisting of an amino acid sequence selected from the group consisting of SEQ ID NOs: 1 to 269 with a cysteine residue added to either or both of an N-terminus and a C-terminus thereof;

3) a method for detecting norovirus comprising using the norovirus-binding peptide of the above 1) or 2); and

4) a norovirus detection kit comprising the norovirus-binding peptide of the above 1) or 2).

### BRIEF DESCRIPTION OF THE DRAWINGS

FIG. 1 is a schematic diagram showing a production flow of VLPs.

FIG. 2 is a schematic diagram showing an outline of screening for norovirus-binding peptides.

FIG. 3 is a schematic diagram showing mRNA-linker conjugates (A: for selection, B: for analysis).

FIG. 4 shows results of evaluation of interaction by ELISA.

### DETAILED DESCRIPTION OF THE INVENTION

The present invention relates to provision of a peptide that specifically binds to norovirus, which is useful for specific detection and infection control of norovirus.

The present inventors constructed a cDNA library containing a 10¹⁴-digit number of cDNAs and succeeded in obtaining peptides that specifically bind to norovirus from the library by a cDNA display method, and accomplished the present invention.

According to the present invention, norovirus-binding peptides having high affinity to norovirus are provided. According to the peptides of the present invention, norovirus can be specifically detected with a high sensitivity, and infection of humans with norovirus can be controlled.

The norovirus-binding peptide of the present invention is a peptide composed of 6 amino acids, consisting of an amino acid sequence selected from the group consisting of SEQ ID NOs: 1 to 269 (Table 16).

The peptides have been screened from the cDNA library containing a 10¹⁴-digit number of cDNAs by a cDNA display method through in vitro selection using norovirus as a target molecule and are norovirus-binding peptide aptamers having an ability of specifically binding to norovirus. The peptides are each composed of 6 amino acids of the library sequences consisting of 34.4% of hydrophobic amino acids and 65.6% of hydrophilic amino acids. The norovirus-binding peptides of the present invention are those that frequently appear (the appearance frequency is six or more in Examples) among norovirus-binding peptides screened by in vitro selection using norovirus as a target molecule. A norovirus-binding peptide consisting of an amino acid sequence selected from the group consisting of SEQ ID NOs: 1 to 101 that more frequently appears (the appearance frequency is eight or more) is preferred. A norovirus-binding peptide consisting of an amino acid sequence selected from the group consisting of SEQ ID NOs: 1 to 52 (the appearance frequency is ten or more) is more preferred, and a norovirus-binding peptide consisting of an amino acid sequence selected from the group consisting of SEQ ID NOs: 1 to 28 (the appearance frequency is 12 or more) is further preferred. A norovirus-binding peptide consisting of an amino acid sequence selected from the group consisting of SEQ ID NOs: 1 to 12, 14 to 17, 19, 21, and 28 is further preferred, and a norovirus-binding peptide consisting of an amino acid sequence selected from the group consisting of SEQ ID NOs: 1, 4, 14, 15, 17, 21, and 28 is further more preferred.

The peptide of the present invention encompasses, as an aspect, a peptide consisting of an amino acid sequence selected from the group consisting of SEQ ID NOs: 1 to 269 with a cysteine residue added to either or both of an N-terminus and a C-terminus thereof. The peptide having cysteine residues at both terminuses can form a cyclic peptide through a disulfide bond of the cysteine residues.

In addition, the peptide of the present invention encompasses, as another aspect, a peptide consisting of an amino acid sequence selected from the group consisting of SEQ ID NOs: 1 to 269, or an amino acid sequence with a cysteine residue added to either or both of the N-terminus and the C-terminus thereof, wherein arbitrary 1 to 20 amino acid residues are further added to either or both of the N-terminus and the C-terminus of the peptide, as long as the ability of specifically binding with norovirus is maintained.

Viruses that belong to norovirus infecting humans are classified into three gene groups of Genogroup I (GI), Genogroup II (GII), and Genogroup IV (GIV) at present, and 90% or more of reported infection cases are in the GII group. It is inferred there is a serotype corresponding to each genotype. In the present invention, the norovirus encompasses viruses belonging to such norovirus. Empty virus-like particles (VLPs) that are extremely similar to virus particles can be produced by incorporating the structural protein region of a norovirus genome into baculovirus and expressing it in insect cells. The VLPs have the structure of norovirus itself and have antigenicity equivalent to that of virus particles, but do not have the genomic RNA therein, being empty and not having infectivity. Accordingly, in the present invention, norovirus encompasses such VLPs.

Examples of the VLPs include VLPs produced using the norovirus genome such as a GII.4 Sagal strain (Genbank No. AB447456), a GII.4 Sydney strain (Genbank No. JX459908.1), a GII.3 TCH strain (Genbank No. KF006265), a GII.2 Ehime strain (Genbank No. LC145808), a GII.17 Kawasaki strain (Genbank No. AB983218), and a GII.17 Saitama strain (Genbank No. KJ196286.1).

The peptide of the present invention can be produced using norovirus (empty virus-like particles: VLPs) as a target molecule by in vitro evolution method known in the art, for example, by a cDNA display method (Nucleic Acid Research, vol. 37, No. 16, e108 (2009)). That is, the peptide can be produced by constructing a cDNA library containing cDNAs (library of peptide-linker-mRNA/cDNA conjugates) and subjecting it to in vitro selection by a cDNA display method.

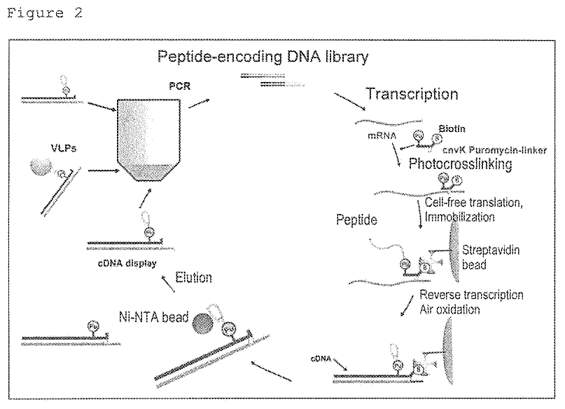

Specifically, the peptide can be produced by the following steps a) to c) (see FIG. 2):

a) a step of preparing DNA fragments (construct) encoding a desired random peptide library;

b) a cDNA display-producing step of producing peptide-linker-mRNA/cDNA in vitro with a cDNA display method using the construct prepared in the above step; and

c) a selection step of mixing the cDNA displays obtained in the above step with VLPs, collecting the cDNA displays bound to the VLPs, and screening for VLP-bound cDNA displays.

a. Step of Preparing Construct

As a construct for producing a norovirus-binding peptide, DNA fragments including a primer region, a promoter region, an untranslated region, a random region, and a tag region from the 5′ end toward the 3′ end and encoding a desired random peptide library are constructed. Here, the DNA sequence used as the primer region may be a commercially available general one. As the promoter region, for example, T7 or SP6 can be used. As the untranslated region, for example, an Ω region can be used.

For the random region, the DNA is constituted such that hydrophobic amino acids of alanine, valine, leucine, isoleucine, methionine, proline, phenylalanine, and tryptophan is 34.4%, polar amino acids of glycine, serine, threonine, asparagine, glutamine, tyrosine, and cysteine is 31.3%, basic amino acids of lysine, arginine, and histidine is 15.6%, and acidic amino acids of aspartic acid and glutamic acid is 6.3%.

b. cDNA Display-Producing Step

The production of cDNA display includes, as shown in FIG. 2, a mRNA preparation step (b1), a linker-mRNA conjugate formation step (b2), a peptide-linker-mRNA conjugate formation step (b3), a particle-binding step (b4), a cDNA display formation step (b5), a peptide crosslinking step (b6), and a cDNA display release step (b7).

In the mRNA preparation step (b1), mRNA is prepared from the above-described construct by transcription. Then, in (b2), a linker-mRNA conjugate is formed by binding the mRNA obtained in the mRNA preparation step to a linker to which puromycin is bound.

Subsequently, in (b3), a peptide-linker-mRNA conjugate is formed by binding a peptide having an amino acid sequence corresponding to the mRNA sequence translated by a cell-free translation system to puromycin.

Subsequently, in the particle-binding step (b4), the peptide-linker-mRNA conjugate obtained as in above is bound to magnetic particles.

Subsequently, in (b5), the mRNA of the peptide-linker-mRNA conjugate bound to the magnetic particles is reversely transcribed to form cDNA to obtain peptide-linker-mRNA/cDNA (“cDNA display”).

Subsequently, in (b6), cysteines on the N-terminus and the C-terminus of the random region of the peptide in the cDNA display obtained in the above step are crosslinked by a crosslinking reaction.

Subsequently, in the complex release step (b7), the cDNA display obtained in the above step is released from the magnetic particles and is purified as needed.

c. Selection Step of VLP-Bound cDNA Displays

The selection of VLP-bound cDNA displays includes a solution addition step (c1), a separation step (c2), and a collection step (c3).

In the solution addition step (c1), a cDNA display-containing solution is added to a VLP solution. Continuously, in the separation step (c2), the mixture solution of the VLP and cDNA display solutions is subjected to, for example, centrifugation at 130,000×g for 5 minutes to precipitate the VLPs. Thus, the cDNA display not bound to the VLPs is separated. Subsequently, in the collection step (c3), the cDNA display bound to the VLPs is collected together with the VLPs.

The peptide of the present invention can be selected from a predetermined DNA library in vitro as described above.

In addition, the peptide of the present invention can be produced by a known method for manufacturing a peptide, for example, by a chemical synthesis method such as a liquid-phase method, a solid-phase method, or a hybrid method of a liquid-phase method and a solid-phase method; or a genetic recombination method.

Since the peptide of the present invention specifically binds to norovirus, it is possible to verify that norovirus is present or not present in a sample by bringing the peptide into contact with the sample that contains or may contain norovirus.

That is, for example, norovirus in a sample can be detected using the peptide of the present invention instead of an anti-norovirus antibody in an immunoassay such as an ELISA method.

The peptide of the present invention when used as a detection reagent may be labeled to be detectable. In labeling of the peptide, for example, not only enzymes such as peroxidase and alkaline phosphatase, but also radioactive materials, fluorescent materials, luminescent materials, etc. are used. In addition, nanoparticles such as colloidal gold and quantum dots, can also be used. In an immunoassay, the peptide of the present invention can also be detected by labeling the peptide with biotin and binding avidin or streptavidin labeled with an enzyme or the like thereto.

Among the immunoassays, an ELISA method using an enzyme label is preferred in the point that it can simply and rapidly measure an antigen. When norovirus is detected by an ELISA method using the peptide of the present invention, for example, norovirus is immobilized on a solid support, and a peptide previously labeled with biotin is bound thereto. After washing, an avidin-modified enzyme is allowed to bind to the biotin and is then allowed to react with an enzyme substrate to cause color development, and the norovirus can be detected by measuring the absorbance. Alternatively, the peptide of the present invention is solid-phased, and norovirus is bound thereto. After washing, an anti-norovirus antibody labeled with an enzyme or an anti-norovirus antibody and an enzyme-labeled secondary antibody is allowed to bind thereto, and the norovirus can be detected by reacting an enzyme substrate to cause color development and measuring the absorbance.

As the enzyme substrate, when the enzyme is alkaline phosphatase, for example, p-nitrophenyl phosphate (NPP) can be used, and when the enzyme is peroxidase, for example, 3,3′,5,5′-tetramethylbenzidine can be used. As the solid support, an insoluble support in a shape of, for example, a bead, microplate, test tube, stick, or test piece made of a material such as polystyrene, polycarbonate, polyvinyl toluene, polypropylene, polyethylene, polyvinyl chloride, nylon, polymethacrylate, latex, gelatin, agarose, cellulose, Sepharose, glass, metal, ceramic, or a magnetic material, can be used. Immobilization of the peptide of the present invention and so on to the solid support can be performed by binding through a known method such as a physical adsorption method, a chemical bond method, or a method of simultaneously performing these methods.

The peptide of the present invention can be a component of a norovirus detection kit. The detection kit can include, in addition to the peptide of the present invention, a reagent and an instrument necessary for detection such as an antibody, a solid support, a buffer solution, an enzyme reaction stopping solution, and a microplate reader.

The sample that is an object of the detection kit is not particularly limited as long as, for example, the sample contains or may contain norovirus, and examples thereof include clinical materials such as feces and vomit collected from a patient, a separated virus culture solution, food such as oyster, and tap and sewage water.

The peptide of the present invention can specifically bind to, for example, the capsid protein of norovirus to inhibit the binding of the virus to a cell. Accordingly, the peptide of the present invention can be used as an anti-norovirus formulation or a medicine for preventing or treating norovirus.

When the peptide of the present invention is used as a medicine, it may be an oral form or a parenteral form and can be appropriately used in combination with known pharmaceutically acceptable avirulent carrier and diluent. Although typical examples of the parenteral administration include an injection, the peptide can also be administered by inhalation with a spray agent, etc.

Regarding the above-described embodiments, the present invention discloses the following aspects:

<1> a norovirus-binding peptide consisting of an amino acid sequence selected from the group consisting of SEQ ID NOs: 1 to 269;

<2> a norovirus-binding peptide consisting of an amino acid sequence selected from the group consisting of SEQ ID NOs: 1 to 269 with a cysteine residue bound to either or both of the N-terminus and the C-terminus thereof;

<3> the norovirus-binding peptide of <2>, wherein the cysteine residue is bound to the N-terminus and the C-terminus of the amino acid sequence selected from the group consisting of SEQ ID NOs: 1 to 269;

<4> the norovirus-binding peptide of <3>, wherein the cysteine residue bound to the N-terminus and the cysteine residue bound to the C-terminus of the peptide consisting of an amino acid sequence selected from the group consisting of SEQ ID NOs: 1 to 269 are linked to each other via a disulfide bond to form a ring;

<5> a norovirus-binding peptide consisting of an amino acid sequence of the peptide according to any one of <1> to <4> with 1 to 20 amino acids bound to either or both of the N-terminus and the C-terminus of the peptide;

<6> a method for detecting norovirus comprising using the norovirus-binding peptide according to any one of <1> to <5>;

<7> a norovirus detection kit comprising the norovirus-binding peptide according to any one of <1> to <5>;

<8> the norovirus-binding peptide according to any one of <1> to <6> or the norovirus detection kit according to <7>, wherein the amino acid sequence selected from the group consisting of SEQ ID NOs: 1 to 269 is an amino acid sequence selected from the group consisting of SEQ ID NOs: 1 to 101;

<9> the norovirus-binding peptide according to any one of <1> to <6> or the norovirus detection kit according to <7>, wherein the amino acid sequence selected from the group consisting of SEQ ID NOs: 1 to 269 is an amino acid sequence selected from the group consisting of SEQ ID NOs: 1 to 52;

<10> the norovirus-binding peptide according to any one of <1> to <6> or the norovirus detection kit according to <7>, wherein the amino acid sequence selected from the group consisting of SEQ ID NOs: 1 to 269 is an amino acid sequence selected from the group consisting of SEQ ID NOs: 1 to 28;

<11> the norovirus-binding peptide according to any one of <1> to <6> or the norovirus detection kit according to <7>, wherein the amino acid sequence selected from the group consisting of SEQ ID NOs: 1 to 269 is an amino acid sequence selected from the group consisting of SEQ ID NOs: 1 to 12, 14 to 17, 19, 21, and 28; and

<12> the norovirus-binding peptide according to any one of <1> to <6> or the norovirus detection kit according to <7>, wherein the amino acid sequence selected from the group consisting of SEQ ID NOs: 1 to 269 is an amino acid sequence selected from the group consisting of SEQ ID NOs: 1, 4, 14, 15, 17, 21, and 28.

### EXAMPLES

The present invention will now be more specifically described by examples.

### Reference Example: Production of VLPs

(1) Introduction of VP1 and VP2 Genes into pDEST8

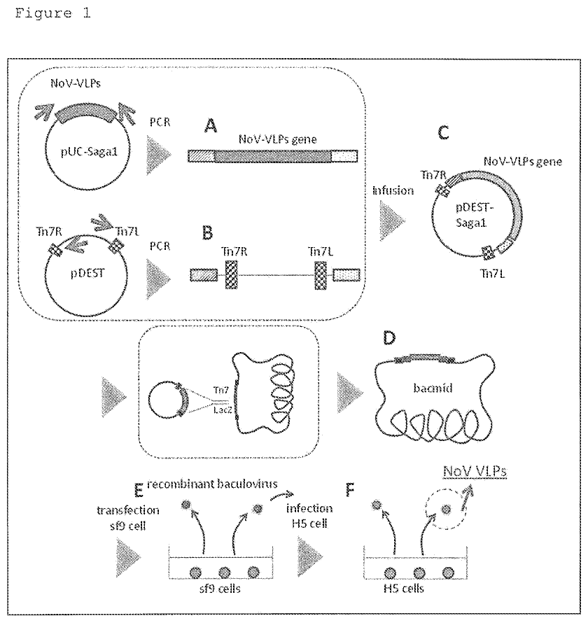

Artificial synthesis of DNAs encoding the VP1 and VP2 regions, which are structural protein regions, of the GII.4 Sagal strain (Genbank No: AB447456), the GII.3 TCH strain (Genbank No. KF006265), and the GII.17 Saitama strain (Genbank No. KJ196286.1) of norovirus (hereinafter, may be abbreviated to NoV), was outsourced to Fasmac Co., Ltd., and a target gene was introduced therein using the position of the slash of 5′-CAGACGTGTGCTCTTCCGATCTGAT/ATCAGATCGGAAGAGCGTCGTTAAG-3′ (SEQ ID NO: 270), which is the lacZ-α region of pUCFa plasmid, as the cloning site. In GII.4, a PCR reaction was performed using a synthesized pUC-Sagal (FIG. 1) DNA as a template and using primer 1 (5′-CATCACAAGTTTGTACAAAAAAGCAGGCTGTGA-3′: SEQ ID NO: 271) and primer 2 (5′-TATCACCACTTTGTACAAGAAAGCTGGGTT-3′: SEQ ID NO: 272) to obtain a fragment (FIG. 1-A). In GII.3, a PCR reaction was performed using a synthesized pUC-TCH DNA as a template and using primer 3 (5′-ATCACAAGTTTGTACTGGGAGGGCGATCGCA-3′: SEQ ID NO: 273) and primer 4 (5′-CTATCACCACTTTGTTCGCTACCTCGCGAA-3′: SEQ ID NO: 274) to obtain a fragment (FIG. 1-A). In addition, a PCR reaction was performed using a pDEST8 plasmid (Invitrogen) as a template and using primer 5 (5′-ACAAGTGGTGATAGCTTGTCGAGAAGTA-3′: SEQ ID NO: 275) and primer 6 (5′-GTACAAACTTGTGATGATCCGCGCCCGAT-3′: SEQ ID NO: 276) to obtain a fragment (FIG. 1-B). The resulting PCR fragment A and fragment B were mixed and were reacted to each other using an InFusion HD Cloning Kit (Clontech Laboratories, Inc.), and using 1 ng of the resulting DNA (FIG. 1-C), Competent Quick DH5α (manufactured by TOYOBO CO., LTD.) was transformed. Selection was performed with an LB agar plate culture medium containing 100 μg/mL of ampicillin, the resulting colonies were cultured in an LB liquid culture medium containing 100 μg/mL of ampicillin, and the plasmid was extracted from the resulting cells using a QIAprep Spin Miniprep Kit (manufactured by QIAGEN N.V.). The sequence of the resulting plasmid with NoV gene introduced was determined using a DNA sequencer to verify that the target sequence was inserted.

In GII.17, a sequence in which an attL1 sequence (5′-ccccaaataatgattttattttgactgatagtgacctgttcgttgcaacaaattgat gagcaatgcttttttataatgccaactttgtacaaaaaagcaggct-3′: SEQ ID NO: 277) was introduced to 4 nucleotides upstream from the start codon of the VP1 region and a polyadenine sequence of 30 adenines followed by an attL2 sequence (5′-agcttacccagctttcttgtacaaagttggcattataagaaagcattgcttatcaat ttgttgcaacgaacaggtcactatcagtcaaaataaaatcattatttg-3′: SEQ ID NO: 278) were introduced to 55 nucleotides downstream from the termination codon of the VP2 region was artificially synthesized. The resulting pUC-Saitama and pDEST8 were mixed in equal amounts, and were mixed as shown in Table 1, followed by a reaction at 25° C. for 1 hour. After the reaction, 1 μL of proteinase K (manufactured by Takara Bio Inc.) was added thereto, followed by a reaction at 37° C. for 10 minutes. Using 1 ng of the reaction solution, Competent Quick DH5α (manufactured by TOYOBO CO., LTD.) was transformed, and selection was performed with an LB agar plate culture medium containing 100 μg/mL of ampicillin. The resulting colonies were cultured in an LB liquid culture medium containing 100 μg/mL of ampicillin, and pDEST8 encoding GII.17 was purified and obtained from the resulting cells using a QIAprep Spin Miniprep Kit (manufactured by QIAGEN N.V.).

TABLE 1

| Composition | Content | |
|---|---|---|
| pUC-Saitama | 0.75 μL | (150 ng) |
| pDEST8 (manufactured by Invitrogen) | 1.0 μL | (150 ng) |
| UltraPure Water (manufactured by | 6.25 | μL |
| Invitrogen) | | |
| BP clonase (manufactured by Thermo | 2 | μL |
| Fisher Scientific) | | |

(2) Introduction of VP1 and VP2 Genes into bMON14272 Bacmid (Manufactured by Invitrogen)

The obtained plasmid was introduced into Bacmid according to the protocol attached to the product by the following method (FIG. 1-D).

NoV VP1 and VP2 regions were introduced into bMON14272 bacmid (manufactured by Invitrogen) using the obtained plasmid having Nov gene introduced and MAX Efficiency DH10Bac Competent Cells (manufactured by Invitrogen) (FIG. 1-D). Whether each gene was introduced into bacmid or not was verified by performing selection in an LB culture medium containing 40 μg/mL of IPTG (isopropyl β-D-1-thiogalactopyranoside: manufactured by FUJIFILM Wako Pure Chemical Corporation), 100 μg/mL of X-Gal (5-bromo-4-chloro-3-indolyl β-D-galactopyranoside: manufactured by FUJIFILM Wako Pure Chemical Corporation), 50 μg/mL of kanamycin, 7 μg/mL of gentamicin, and 10 μg/mL of tetracycline (kanamycin resistance gene, tetracycline resistance gene, and gentamicin resistance gene were encoded in bMON14272, helper plasmid present in DH10Bac Competent Cell, and the region of pDEST to be inserted into bacmid, respectively), and further whether each fragment was inserted into a target side or not was verified by color selection. The obtained white colonies were cultured in an LB liquid culture medium containing 50 μg/mL of kanamycin, 7 μg/mL of gentamicin, and 10 μg/mL of tetracycline, and bacmid was extracted from the resulting cells using a QIAprep Spin Miniprep Kit (manufacture by QIAGEN N.V.). The concentration of the extracted DNA solution was verified with NanoDrop (manufactured by Thermo Fisher Scientific).

(3) Production of Recombinant Baculovirus (rBV) by Transfection of Bacmid Having NoV VP1 and VP2 Introduced

Bacmid into which NoV VP1 and VP2 genes were introduced was transfected into Sf9 cells (manufactured by Invitrogen) using a Lipofecctamine LTX Reagent & Plus Reagent (manufactured by Invitrogen) according to the protocol attached thereto (FIG. 1-E). The transfected cells were cultured using an Sf900III (manufactured by Invitrogen) culture medium at 27° C. for 1 week. After the culture, the culture medium was centrifuged, and the supernatant was collected to obtain recombinant baculovirus (rBV) including the NoV gene.

(4) Production of NoV VLP by Infection with rBV

The rBV was added at 1.0×10⁷ pfu/mL to 1.0×10⁷ cells/flask of High Five cells (manufactured by Invitrogen) to cause infection at an MOI of 2, and the cells were cultured using an Express five (manufactured by Invitrogen) culture medium at 27° C. (FIG. 1-F). After 7 days from the infection, the culture supernatant was collected by centrifugation. The collected supernatant was further centrifuged at 10,000×g for 1 hour to pellet down the baculovirus, and the cell supernatant was collected. The collected supernatant containing NoV VLP was further centrifuged with an SW32Ti rotor (manufactured by Beckman Coulter, Inc.) at 32,000 rpm for 2 hours to pellet down the NoV VLP. The pellet separated from the supernatant was dissolved in an Express five culture medium containing 1.9 mg of CsCl (for density gradient centrifugation, manufactured by FUJIFILM Wako Pure Chemical Corporation) and centrifuged using SW55Ti (Manufactured by Beckman Coulter, Inc.) an at 40,000 rpm for 20 hours for separation and purification, and a fraction visually observed by irradiation with white light was collected. The collected fraction was centrifuged again with the SW32Ti rotor at 32,000 rpm for pellet down, the supernatant was removed, and the pellet was suspended in 500 μL of an Express five culture medium. The VLP concentration was quantitatively measured by a Bradford method. As a standard protein, BSA (manufactured by FUJIFILM Wako Pure Chemical Corporation) was used.

### Example 1: Manufacturing of Norovirus-Binding Peptide

### (see FIG. 2)

### (1) Construction of DNA Library

A DNA library was designed such that the peptide library is composed of peptides having a length of 10 amino acids.

### (Nucleotide Sequence of DNA Library)

(SEQ ID NO: 279)
5'-GATCCCGCGAAATTAATACGACTCACTATAGGGGAAGTATTTTTACA
ACAATTACCAACAACAACAACAAACAACAACAACATTACATTTTACATTC
TACAACTACAAGCCACCATGGGCAGCGGCGGCAGCTGCNNKNNKNNKNNK
NNKNNKTGCGGGGGAGGCAGCCATCATCATCATCATCACGGCGGAAGCAG
GACGGGGGGCGGCGTGGAAA-3'

TABLE 2
Each sequence included in template DNA and Position thereof

| Nucleotide No. | Region Name |
|---|---|
| 14 to 33 | T7 promoter |
| 34 to 36 | 5′ cap |
| 37 to 107 | Ω sequence |
| 110 to 114 | Kozak sequence |
| 115 to 132 | MGSGGS |
| 133 to 156 | Cys-library sequence-Cys |
| 157 to 168 | GGGS |
| 169 to 186 | Hexahistidine tag |
| 187 to 195 | GGS |
| 196 to 217 | Hybridization region for linker DNA |

The above-mentioned DNA library was constructed by binding three DNA sequence fragments, a T7-PRO-Ω region (SEQ ID NO: 280), a random region (SEQ ID NO: 281), and a His-Y tag region (SEQ ID NO: 282), by extension PCR. This library was designed such that cysteines appear on the N-terminus and the C-terminus of the random region. The random region, the His-Y tag region, and the T7-PRO-Ω region were obtained by outsourcing the respective DNA synthesis to TSUKUBA OLIGO SERVICE CO. LTD. The following extension PCR was performed using them to construct the above DNA library.

[SEQ ID NO: 280]
5′ GATCCCGCGAAATTAATACGACTCACTATAGGGGAAGTATTTTTACA
ACAATTACCAACAACAACAACAAACAACAACAACATTACATTTTACATTC
TACAACTACAAGCCACCATG 3′
[SEQ ID NO: 281]
5′-ACAACTACAAGCCACCATGGGCAGCGGCGGCAGCTGCNNKNNKNNKN
NKNNKNNKTGCGGGGGAGGCAGCCATCATCA-3′

In the sequences above, the ratios of appearance frequency of nucleotides (A:T:G:C) other than ATCG are as follows:

### N: A=0.25, T=0.25, G=0.25, C=0.25, and

### K: A=0, T=0.5, G=0.5, C=0.

[SEQ ID NO: 282]
5′ TTTCCACGCCGCCCCCCGTCCTGCTTCCGCCGTGATGATGATGATGA
TGGCTGCCTCCCCC 3′

In the extension PCR of the first stage of synthesis, a reaction solution having the composition shown in the following Table 3 was prepared to 50 μL with ultrapure water, and a DNA fragment including the random region and the His-Y tag region bound to each other was amplified by the following PCR program. The PCR program included (a) 96° C. (2 min), (b) 94° C. (20 sec), (c) 69° C. (5 sec), (d) 72° C. (20 sec), and (e) 72° C. (2 min), and the steps (b) to (d) were repeated 5 cycles.

TABLE 3

| Composition | Content (μL) |
|---|---|
| Random region (10 pmol/μL) | 1 |
| His-Y tag region (10 pmol/μL) | 1 |
| 5 × PrimeSTAR Buffer (manufactured by | 10 |
| Takara Bio Inc.) | |
| dNTP mixture (25 mM each) (manufactured by | 4 |
| Takara Bio Inc.) | |
| TaKaRa PrimseSTAR (manufactured by | 0.5 |
| Takara Bio Inc.) | |

In the extension PCR of the second stage, a reaction solution having the composition shown in the following Table 4 was prepared to 50 μL with ultrapure water, the T7-PRO-SD region was extended by the following PCR program to amplify the DNA library. The PCR program included (a) 96° C. (2 min), (b) 94° C. (20 sec), (c) 59° C. (5 sec), (d) 72° C. (30 sec), and (e) 72° C. (2 min), and the steps (b) to (d) were repeated 15 cycles. Subsequently, the DNA library was purified by polyacrylamide gel electrophoresis (PAGE).

TABLE 4

| Composition | Content (μL) |
|---|---|
| Extended PCR product in first stage | 10 |
| (0.5 pmol/μL) | |
| T7-PKO-Ω region (10 pmol/μL) | 5 |
| 5 × PrimeSTAR Buffer (manufactured by | 10 |
| Takara Bio Inc.) | |
| dNTP mixture (25 mM each) (manufactured by | 4 |
| Takara Bio Inc.) | |
| TaKaRa PrimeSTAR (manufactured by | 0.25 |
| Takara Bio Inc.) | |

### (2) Transcription of DNA Library

Transcription of the DNA library was performed using RiboMAX Large Scale RNA Production Systems-T7 (manufactured by Promega Corporation) according to the protocol attached thereto. The reaction scale was 20 μL using 1 μg of the DNA library. The mRNA obtained by the transcription reaction was purified using an After Tri-Reagent RNA Clean-Up Kit (manufactured by FAVORGEN Biotech Corporation).

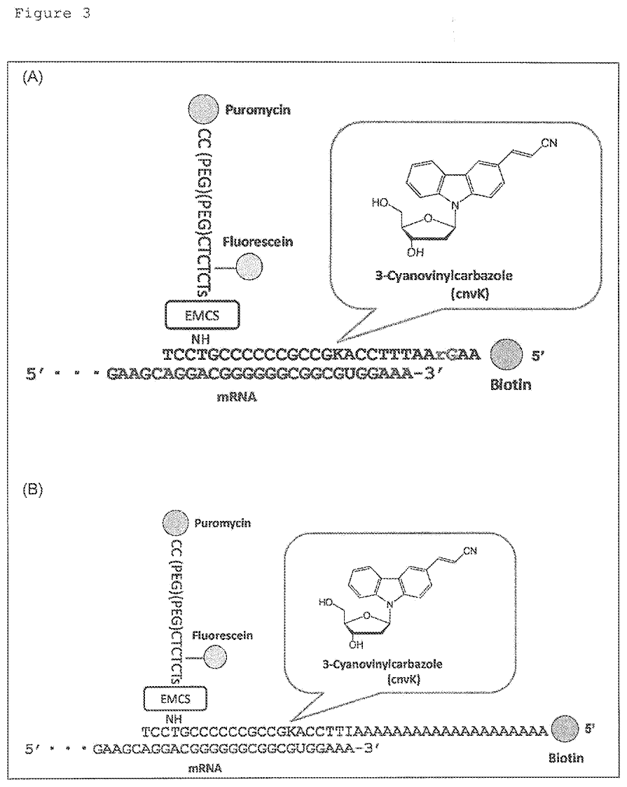

Subsequently, the obtained mRNA was ligated to a puromycin linker described later as follows (FIG. 3 (A)). Firstly, 20 pmol of each of a puromycin linker and the mRNA, 4 μL of 0.25 M Tris-HCl (pH 7.5), and 4 μL of 1 M NaCl were mixed, and the mixture was diluted to 20 μL with ultrapure water. The reaction solution was incubated at 90° C. for 2 minutes and at 70° C. for 1 minute, was then cooled to 4° C., and was then annealed at 25° C. for 1 hour. Subsequently, crosslinking with the puromycin linker was performed using a CL-1000 Ultraviolet Crosslinker by irradiation with ultraviolet light having a wavelength of 365 nm under a condition of 405 mJ/cm².

### <DNA of Puromycin Linker>

Puromycin linker DNA 1 (FIG. 3 (A)) was synthesized by chemical crosslinking of two segments (puromycin segment (PS) and a short biotin segment (SBS)) using EMCS (N-(6-maleimidocaproyloxy)succinimide: manufactured by DOJINDO LABORATORIES). The linker used was that described in the literature (Mochizuki Y., Suzuki T., Fujimoto K., Nemoto N., (2015), A versatile puromycin-linker using cnvK for high-throughput in vitro selection by cDNA display, J. Biotechnol., 212, 174-80).

The sequence structure of the puromycin segment (PS) is shown below:

5′-(S)-TCTCTC(F)-(PEG)(PEG)-CC-(Puro)-3′.

Here, (S) represents 5′-thiol-modifier C6 (compound name: S-trityl-6-mercaptohexyl-1-[(2-cyanoethyl)-(N,N-diisopropyl)]-phosphoramidite, manufactured by Glen research), and (F) represents fluoresceine-dT. (Puro) represents puromycin CPG (5′-dimethoxytrityl-N-trifluoroacetyl-puromycin,2′-succinoyl-long chain alkylamino-CPG, manufactured by Glen research).

The sequence structure of the short biotin segment (SBS) is then shown below:

(SEQ ID NO: 283)
5′(B)-AA-(rG)-AATTTCCA(K)GCCGCCCCCCG(Y)CCT-3′.

Here, (Y) represents amino-modifier C6 deoxythymine (5′-dimethoxytrityl-5-[N-(trifluoroacetylaminohexyl)-3-acrylimido]-2′-deoxyuridine,3′-[(2-cyanoethyl)-(N,N-diisopropyl)]-phosphoramidite, manufactured by Glen research), (K) represents 3-cyanovinylcarbazole (cnvK), (B) represents biotin-triethylene glycol (TEG), manufactured by Glen research), and (rG) represents riboguanine (manufactured by Glen research). Synthesis of PS and SBS was outsourced to TSUKUBA OLIGO SERVICE CO. LTD. and was performed according to a usual method.

The 5′-thiol group of the PS was reduced with 1 mM tris(2-carboxyethyl)phosphine hydrochloride (TCEP: manufactured by Thermo Fisher Scientific) in 100 μL of a 50 mM phosphate buffer (pH 7.0) at room temperature for 6 hours, and desalted with a NAP-5 column (manufactured by GE Healthcare) at the time of use. A biotin loop in a total amount of 10 nmol and EMCS in a total amount of 2 μmol were added to 100 μL of a 0.2 M sodium phosphate buffer (pH 7.0). Subsequently, the mixture was incubated at 37° C. for 30 minutes, and ethanol precipitation was performed at 4° C. to remove excess EMCS.

This precipitate was washed twice with 500 μL of 70% ethanol cooled in advance in an ice bath and was dissolved in 10 μL of a 0.2 M sodium phosphate buffer (pH 7.0) cooled in advance. The reduced PS was immediately added thereto, followed by stirring at 4° C. overnight. After addition of 4 mM TCEP, incubation was performed at 37° C. for 15 minutes to stop this reaction. Subsequently, ethanol precipitation was performed to remove excess PS at room temperature. In order to remove the biotin loop and uncrosslinked biotin loop-EMCS complex, the precipitate was dissolved in a 0.1 M TEAA (triethylamine acetate: manufactured by Glen research) or phosphate buffer and was purified using a C18 HPLC column under the following condition:

Column: AR-300, 4.6×250 mm (manufactured by NACALAI TESQUE, INC.); solvent: A: 0.1 M TEAA, solvent B: acetonitrile/water (80:20, v/v) gradient, B/A (15 to 35%, 33 min); flow rate: 0.5 mL/min; and detection wavelength: absorbance 254 nm and 490 nm.

Fractions from the final peak at absorbance 254 nm (corresponding to a single peak at absorbance 490 nm) were collected. After drying, the fractions were resuspended in water treated with diethylpyrocarbonate (DEPC) and were stored. As described above, puromycin linker DNA 1 could be obtained.

<Binding of mRNA and Puromycin Linker DNA 1>

To 20 pmol of the mRNA obtained by transcription, 20 pmol of the puromycin linker DNA 1, 4 μL of 0.25 M Tris-HCl (pH 7.5), and 4 μL of 1 M NaCl were added, and prepared to 20 μL in total with nuclease-free water (Table 5). Incubation was performed at 90° C. for 1 minute and then at 70° C. for 1 minute, and the temperature was then lowered to 25° C. at a rate of 0.04° C./s. The cnvK and uracil in the mRNA were covalently bonded by irradiation with 405 mJ of ultraviolet light (365 nm) to form a mRNA-linker conjugate. The amount synthesized here was that required in each round.

TABLE 5
Composition for binding of mRNA and puromycin linker DMA

| Composition | Content | |
|---|---|---|
| Puromycin linker DNA 1 | 20 | pmol |
| mRNA | 20 | pmol |
| 0.25M Tris-HCl (pH 7.5) | 4 | μL |
| 1M NaCl | 4 | μL |

### <Translation>

The mRNA-linker conjugate was translated by a cell-free translation system as follows. A reaction solution having the composition ratio shown in the following Table 6 was prepared to 50 μL with ultrapure water and was reacted at 37° C. for 15 minutes, and 24 μL of 3 M KCl and 6 μL of 1 M MgCl₂ were added to this reaction solution. Subsequently, this solution was further reacted at 37° C. for 20 minutes to bind between the C-terminus of the translated peptide and puromycin to obtain a mRNA-peptide conjugate.

TABLE 6

| Composition | Content | |
|---|---|---|
| Rabbit reticulocyte lysate, nuclease treated | 35 | μL |
| (manufactured by Promega Corporation) | | |
| Amino acid mixture minus leucine, 1 mM | 0.5 | μL |
| (manufactured by Promega Corporation) | | |
| Amino acid mixture minus cysteine, 1 mM | 0.5 | μL |
| (manufactured by Promega Corporation) | | |
| mRNA/linker ligation product | 6 | pmol |

### (3) Purification by Magnetic Beads

Streptavidin (SA) magnetic particles (Dynabeads MyOne Streptavidin C1, manufactured by Invitrogen) were washed according to the manual and were put in an Eppendorf tube in an amount required for immobilizing the peptide-linker-mRNA conjugate, followed by leaving to stand on a magnetic stand for 1 minute. Subsequently, the supernatant was removed, followed by resuspension in a solution A (100 mM NaOH, 50 mM NaCl). After tapping for 1 to 2 minutes, the tube was left to stand on a magnetic stand for 1 minute. Subsequently, the same operation was repeated once with the solution A, and the same operation was repeated once with a solution B (100 mM NaCl).

To the peptide-linker-mRNA conjugate, the same amount of 2×binding buffer (20 mM Tris-HCl (pH 8.0), 2 mM EDTA, 2 M NaCl, 0.2% Tween 20, and 500 mM EDTA) was added, and the mixture was incubated together with the streptavidin (SA) magnetic particles at room temperature for 30 minutes. The Eppendorf tube was left to stand on a magnetic stand for 1 minute, and the supernatant was then removed. After addition of 200 μL of 1×binding buffer, tapping was performed for 1 to 2 minutes, and the tube was then left to stand on a magnetic stand for 1 minute, followed by removal of the supernatant. This operation was further repeated twice to immobilize the peptide-linker-mRNA conjugate on the streptavidin (SA) magnetic particles.

(4) Synthesis of cDNA by Reverse Transcription Reaction

A reaction solution of the ratio shown in the following Table 7 was added to the immobilized peptide-linker-mRNA conjugate in the same volume as that of the streptavidin (SA) magnetic particles, and incubation was performed at 42° C. for 30 minutes for reverse transcription to prepare cDNA display in the state that the conjugate was immobilized on the streptavidin (SA) magnetic particles.

TABLE 7

| Composition | Content (μL) |
|---|---|
| 2.5 mM dNTP MIX (manufactured by | 20 |
| Takara Bio Inc.) | |
| 5 × RT Buffer (manufactured by | 10 |
| TOYOBO CO., LTD.) | |
| Nuclease-free water | 18 |
| ReverTra Ace (manufactured by | 2 |
| TOYOBO CO., LTD.) | |

### (5) Crosslinking Reaction of Peptide

The cDNA display immobilized on the streptavidin (SA) magnetic beads were washed with a crosslinking buffer (containing 100 mM sodium phosphate (pH 7.4), 0.15 M NaCl, 10 mM EDTA, and 0.025% Tween 20) once, and then 125 μL of a crosslinking buffer containing 10 mM tris(2-carboxyethyl)phosphine hydrochloride (TCEP: manufactured by Thermo Fisher Scientific) and 4 mM bismaleimidoethane (BMOE: manufactured by Thermo Fisher Scientific) was added, followed by incubation at 25° C. for 1 hour to perform crosslinking reaction of the cysteines on the N-terminus and the C-terminus of the random region.

(6) Elution from Purification by Magnetic Beads

The cDNA display immobilized on the streptavidin (SA) magnetic beads was washed with 1×His-tag wash buffer (containing 10 to 30 mM sodium phosphate (pH 7.4), 0.25 to 0.75 M NaCl, 5 to 30 mM imidazole, and 0.025% to 0.1% Tween 20) once, and then 30 μL of 1×His-tag wash buffer containing 10 U of RNase T1 (manufactured by Ambion, Inc.) was added, followed by incubation at 37° C. for 15 minutes to elute the cDNA display cleaved from the streptavidin (SA) magnetic beads at the cleavage site (ribo G) in the liker.

### (7) Purification by Ni-NTA

Ni-NTA magnetic beads (His Mag Sepharose Ni: manufactured by GE Healthcare) were put at 10 μL in an Eppendorf tube, followed by leaving to stand on a magnetic stand for 1 minute. Subsequently, the supernatant was removed, followed by resuspension in 1×His-tag wash buffer. Tapping was performed for 1 to 2 minutes, and the tube was then left to stand on a magnetic stand for 1 minute. This procedure was further repeated once more.

The cDNA display was incubated together with the Ni-NTA magnetic beads at room temperature for 30 minutes. The Eppendorf tube was left to stand on a magnetic stand for 1 minute, and the supernatant was then removed. After addition of 200 μL of 1×His-tag wash buffer, tapping was performed for 1 to 2 minutes, and the tube was then left to stand on a magnetic stand for 1 minute, followed by removal of the supernatant. This operation was further repeated, and 10 μL of a His-tag elution buffer (containing 10 to 30 mM sodium phosphate (pH 7.4), 0.25 to 0.75 M NaCl, 250 to 500 mM imidazole, and 0.025% to 0.1% Tween 20) was then added, followed by incubation at room temperature for 15 minutes to purify the cDNA display.

### (8) In Vitro Selection Cycle

The cDNA displays and VLPs were mixed according to the following Table 8 and were prepared to 1 mL with a selection buffer (containing 10 to 30 mM Tris-HCl (pH 7.4) and 0.1 to 0.3 M NaCl), followed by incubation at 25° C. for 30 minutes.

TABLE 8

| | cDNA display | | VLP |
|---|---|---|---|
| R1 | 100 | nM | GII.4 500 nM |
| R2 | 16 | nM | GII.4 50 nM |
| R3 | 10 | nM | GII.4 25 nM |
| R4 | 4 | nM | GII.4 25 nM |
| R5 | 4 | nM | GII.4 20 nM |
| R6 | 4 | nM | GII.4 10 nM |
| R7 | 4 | nM | GII.4 10 nM |

R1 to R7 are the numbers of in vitro selection cycles.

### <Separation of VLP-cDNA Display Complex by Centrifugation>

The above mixture was put in a centrifuge tube and was centrifuged with an ultracentrifuge (CS150FNX, manufactured by Hitachi, Ltd.) at 130,000×g at 4° C. for about 2 hours. The supernatant was removed. The wall surface was washed with 1 mL of a selection buffer, and the supernatant was then removed. The precipitate was redissolved in 100 μL of RNase-free water.

### <Separation of VLP-cDNA Display Complex by Dialysis>

The constructed cDNA display and VLPs were incubated in 100 μL of a dialysis selection buffer (20 mM HEPES (pH 7.4), 150 mM NaCl, and 0.05% s Tween 20) at the concentrations shown in Table 9 below at 25° C. for 30 minutes. Subsequently, the resultant was diluted to 1 mL with the dialysis selection buffer, and was put in Float-A-Lyzer G2 Dialysis Device CE, 1000kD MWCO (manufactured by Spectrum Laboratories, Inc.) and was dialyzed with 1 L of the dialysis selection buffer as the external solution at 25° C. During the dialysis, the external solution was replaced with new one 3 times every 2 hours, and the dialysis was performed overnight (for 8 hours) after the 4th replacement. The dialysis product was concentrated with Amicon Ultra 100K (manufactured by Merck Millipore S.A.S.) at 14,000×g for 5 minutes.

### (9) Selection of GII.3 and GII.17 VLP as Objects

To an immunoplate (C-BOTTOM, CLEAR, MICROLON (registered trademark), HIGH BINDING, manufactured by Greiner Bio-One), 100 μL of 3 μg/mL GII.3 or GII.17 VLP solution was added and immobilization was performed at 4° C. overnight. Subsequently, the solution was discarded, and 200 μL of a blocking agent (EMD Millipore™ Blok™ NSB Blocking agents, Thermo Fisher Scientific) was added, followed by gently stirring at room temperature for 2 hours for blocking. On this occasion, wells not immobilizing the VLP were also subjected to similar blocking to be used in preselection.

The solution was discarded from the wells, the wells were washed with 200 μL of a wash buffer (10 mM Hepes (pH 7.4), 150 mM NaCl, and 0.05% Tween 20) three times, and 100 μL of cDNA display (constructed from 1.5 pmol of mRNA-linker) was then put in the wells not immobilizing the VLP, followed by gently stirring at room temperature for 30 minutes to perform preselection. Subsequently, the supernatant containing cDNA display that had not bound to the blocking agent was put in the wells immobilizing the VLP, followed by gently stirring at room temperature for 30 minutes to be bound to the VLP. The supernatant was discarded, washing with 100 μL of the wash buffer was performed four times, and 100 μL of a 5′ SDS solution was then added, followed by incubation at 50° C. for 15 minutes to elute the bound cDNA display.

Subsequently, the VLP-cDNA display complex obtained above was diluted to 100 μL with a dialysis selection buffer, and 10 μL of a coprecipitating agent (Quick-Precip Plus Solution, manufactured by EdgeBio) and 220 μL of 100% ethanol were added, followed by centrifugation at 20,000×g for 5 minutes. Subsequently, the supernatant was discarded, and 1 mL of 70, ethanol was added for rinsing. The tube was dried for 10 minutes, elution with 20 μL of RNase-free water was then performed, and PCR reaction was performed using GATCCCGCGAAATTAATACGACTCACTATAGGGGAAGTATTTTTACAACAATTACCA ACA (SEQ ID NO: 284) as a forward primer and TTTCCACGCCGCCCCCCGTCCT (SEQ ID NO: 285) as a reverse primer. The PCR program was (a) 98° C. for 2 minutes, (b) 95° C. for 20 seconds, (c) 69° C. for 20 seconds, (d) 72° C. for 20 seconds (steps (b) to (d) were performed 25 cycles), and (e) 72° C. for 1 minute.

TABLE 9

| Composition | Content (μL) |
|---|---|
| 10 × Ex Taq Buffer (manufactured by | 2.5 |
| Takara Bio Inc.) | |
| 2.5 mM dNTP mixture (manufactured by | 2 |
| Takara Bio Inc.) | |
| 20 μM forward primer (SEO ID NO: 284) | 0.5 |
| 20 μM reverse primer (SEO ID NO: 285) | 0.5 |
| Ethanol precipitate | 3 |
| Nuclease-free water | 16.4 |
| Ex Taq | 0.1 |

The resulting PCR product was used as library DNA in the subsequent cycle, and the operations after the transcription of library described in the above (2) were similarly performed to repeat a selection cycle.

### <Analysis of Genetic Sequence Information>

After the in vitro selection cycle (7 cycles for GII.4 and 5 cycles for GII.3 and GII.17 based on the libraries of 7 cycles obtained by GII.4 dialysis), a sequence library was prepared by the following method, and the sequence information was analyzed. The preparation of the sequence library and the sequencing were performed according to the 16S Metagenomic Sequencing Library Preparation protocol (manufactured by Illumina, Inc.).

### 1) Amplicon PCR

The reagents shown in Table 10 were mixed, and PCR was performed by the following program:

at 95° C. for 3 minutes;

23 cycles of the following reactions;
at 95° C. for 30 seconds,
at 55° C. for 30 seconds, and
at 72° C. for 30 seconds,

at 72° C. for 5 minutes; and

holding at 4° C.

TABLE 10
Composition of solution

| | |
|---|---|
| Sequence library (5 ng/μL) | 2.5 μL |
| Amplicon PCR Forward Primer 1 μM (SEQ ID NO: 286) | 5 μL |
| Amplicon PCR Reverse Primer 1 μM (SEQ ID NO: 287) | 5 μL |
| 2 × KAPA HiFi Hotstart ReadyMix (manufactured by NIPPON Genetics Co., Ltd,) | 12.5 μL |
| Total | 25 μL |

SEQ ID NO 286: TCGTCGGCAGCGTCAGATGTGTATAAGAGACAGCATTCTACAACTACAAGCCACCATG
SEQ ID NO 287: GTCTCGTGGGCTCGGAGATGTGTATAAGAGACAGTTTCCACGCCGCCCCCCGTCCTGCTTC

### 2) Clean Up

The Amplicon PCR product was purified using AMPure XP beads (manufactured by Beckman Coulter, Inc.). To the plate including the PCR product, 20 μL of the AMPure XP beads were added and the mixture was gently mixed by pipetting with a micropipette 10 times, followed by leaving to stand at room temperature for 5 minutes. The plate was placed on a magnetic stand and was left to stand for 2 minutes, and the supernatant was then discarded. While the plate was being placed on the magnetic stand, 200 μL of 80% ethanol was added to each well, and after leaving to stand for 30 seconds, the supernatant was discarded. This procedure was repeated twice. The ethanol was air-dried by leaving to stand for 10 minutes, the plate was then taken out from the magnetic stand, and 52.5 μL of 10 mM Tris pH 8.5 solution was added to each well, followed by leaving to stand at room temperature for 2 minutes. The plate was placed on the magnetic stand and was left to stand for 2 minutes again, and 50 μL of the solution in each well was transferred to the corresponding well of a new 96-well PCR plate.

### 3) Index PCR

A PCR reaction was performed for adding an adaptor and an index sequence for sequencing to the purified Amplicon PCR product.

The reagents shown in Table 11 were mixed, and PCR was performed by the following program:

at 95° C. for 3 minutes;

8 cycles of the following reactions;
at 95° C. for 30 seconds,
at 55° C. for 30 seconds, and
at 72° C. for 30 seconds,

at 72° C. for 5 minutes; and

holding at 4° C.

TABLE 11
Composition of solution

| | | |
|---|---|---|
| Purified Amplicon PCR product | 2.0 | μL |
| Nextera XT Index Primer 1 (N7xx) | 2.5 | μL |
| (SEQ ID NOs: 288 and 289) | | |
| Nextera XT Index Primer 2 (S5xx) | 2.5 | μL |
| (SEQ ID NOs: 290 and 291) | | |
| 2x KAPA HiFi Hotstart ReadyMix | 12.5 | μL |
| UltraPure Water (manufactured by | 5 | μL |
| Invitrogen) | | |
| Total | 25 | μL |

The used Index Primer set is shown in the following Table 12.

TABLE 12
Index Primer set

| | Centrifugation | Dialysis |
|---|---|---|
| Run 1 (S511) | S511, N724 | S511, N728 |
| Run 2 (S510) | S510, N724 | S510, N728 |

SEQ ID NO: 288 (N724):
CAAGCAGAAGACGGCATACGAGATACTGAGCGGTCTCGTGGGCTCGG
SEQ ID NO: 289 (N728):
CAAGCAGAAGACGGCATACGAGATTGCAGCTAGTCTCGTGGGCTCGG
SEQ ID NO: 290 (S511):
AATGATACGGCGACCACCGAGATCTACACTCTCTCCGTCGTCGGCAGCGTC
SEQ ID NO: 291 (S510):
AATGATACGGCGACCACCGAGATCTACACCGTCTAATTCGTCGGCAGCGTC

### 4) Clean Up 2

The Index PCR product was purified using AMPure XP beads (manufactured by Beckman Coulter, Inc.). To the plate including the PCR product, 56 μL of the AMPure XP beads were added and the mixture was gently mixed by pipetting with a micropipette 10 times, followed by leaving to stand at room temperature for 5 minutes. The plate was placed on a magnetic stand and was left to stand for 2 minutes, and the supernatant was then discarded. While the plate was being placed on the magnetic stand, 200 μL of 80% ethanol was added to each well, and after leaving to stand for 30 seconds, the supernatant was discarded. This procedure was repeated twice. The ethanol was air-dried by leaving to stand for 10 minutes, the plate was then taken out from the magnetic stand, and 25 μL of 10 mM Tris pH 8.5 solution was added to each well, followed by leaving to stand at room temperature for 2 minutes. The plate was placed on the magnetic stand and was left to stand for 2 minutes again, and 50 μL of the solution in each well was transferred to the corresponding well of a new 96-well PCR plate.

The purified Index PCR product was validated using Bioanalyzer DNA 1000 Chip (manufactured by Agilent Technologies, Inc.).

5) qPCR

The purified Index PCR product was subjected to qPCR using Kapa Library Quantification Kit (manufactured by NIPPON Genetics Co., Ltd.).

A mixture of 12 μL of Kapa SYBR FAST qPCR Master Mix to which Primer Mix was added in advance, 4 μL of UltraPure Water, and 4 μL of a 100-fold dilution of the Index PCR product was subjected to qPCR. As the samples for a standard curve, Std 1 to 6 included in the kit were used.

The PCR was performed by the following program:

at 95° C. for 5 minutes; and

35 cycles of the following reactions;
at 95° C. for 30 seconds, and
at 60° C. for 45 seconds.

A standard curve was drawn from the Ct values of Std 1 to 6, and sample concentration was calculated.

### 6) Preparing DNA Libraries for Sequencing

The Reagent Cartridge of Miseq Reagent Kit V3 150 cycles (manufactured by Illumina, Inc.) was thawed in a water bath, and the HT1 buffer included in the kit was thawed at room temperature and ice-cooled.

The Index PCR product having a concentration known by qPCR was diluted to 4 nM with UltraPure Water. A mixture of 5 μL of this 4 nM dilution of the sample and 5 μL of 0.2 N NaOH (prepared by diluting 10 N NaOH aqueous solution (manufactured by FUJIFILM Wako Pure Chemical Corporation) to 0.2 N with UltraPure Water) was left to stand at room temperature for 5 minutes. Subsequently, 990 μL of ice-cooled HT1 buffer was added thereto to obtain 1 mL of a 20 pM denatured library.

A mixture of 180 μL of the 20 pM denatured library and 420 μL of ice-cooled HT1 buffer was prepared as 600 μL of a 6 pM library. In addition, a mixture of 30 μL of a 20 pM PhiX DNA denatured in advance and 10 μL of ice-cooled HT1 buffer was prepared as a 15 pM denatured PhiX. A mixture of 30 μL of the 15 pM denatured PhiX and 570 μL of the 6 pM denatured library in total of 600 μL was added to “Load Samples” (position 17) of the Reagent Cartridge thawed in a water bath.

### 7) Starting the Run

Flow Cell washed with Milli-Q water and 99.5, ethanol was set to Miseq (manufactured by Illumina, Inc.) subjected to Maintainance Wash with 0.5% Tween 20, and a PR2 bottle and a reagent-filled cartridge were set, followed by sequencing.

### 8) Analysis of Gene Information

The Fastq file of the obtained sequence was converted to a Fasta file, all of the obtained sequences were simultaneously translated from the first base of the start codon (ATG) at position 115 to the third base of the cysteine codon (TGC) at position 156 of the library sequence (SEQ ID NO: 279) using software MEGA. After the translation, the amino acid 7 residues upstream from the terminal cysteine was filtered with cysteine using the filter function of Excel to obtain 4357 peptide aptamer sequences.

### 9) Selection of Sequence

The obtained 7069 peptide aptamer sequences were subjected to appearance frequency analysis, and the peptides shown in SEQ ID NOs: 1 to 269, which are sequences having an appearance frequency of 6 or more, were selected as peptides that specifically bind to norovirus. The selected 269 sequences are shown in Table 16.

### Example 2: Interaction with VLPs

### <Synthesis of Peptide>

Peptides were synthesized by Fmoc solid synthesis in a nitrogen atmosphere using an automated peptide synthesizer Liberty Blue (manufactured by CEM Corporation). The resin used was Fmoc-Lys (Mtt)-Wang resin (manufactured by Merck Millipore S.A.S.) or Fmoc-Cys (Trt)-Wang Resin (manufactured by PEPTIDE INSTITUTE, INC.). N,N-Dimethylformamide: DMF (manufactured by FUJIFILM Wako Pure Chemical Corporation) was used as a solvent, and piperidine (manufactured by FUJIFILM Wako Pure Chemical Corporation) diluted with DMF to a predetermined concentration was used as a deprotecting agent. Diisopropylcarbodiimide (manufactured by Tokyo Chemical Industry Co., Ltd.) and ethyl cyanoglyoxylate-2-oxime: Oxyma (manufactured by WATANABE CHEMICAL INDUSTRIES, LTD.) diluted with DMF to predetermined concentrations were used as a coupling reaction accelerator and an optical activity inhibitor, respectively. The synthesis reaction was performed according to the synthesis program provided in the apparatus.

### <Introduction of Biotin by Manual Synthesis>

A synthesis reaction was performed using a manual peptide synthesizer Petisyzer (manufactured by HiPep Laboratories). Biotin was introduced to the C-terminus side of the tryptophan by using a mixture solution prepared at the following ratio (Table 13) and stirring at room temperature for 1 hour. After the reaction, washing with DMF and diethyl ether was performed.

TABLE 13

| Reagent name | Equivalent |
|---|---|
| HBTU | 5 |
| HOBt | 4.5 |
| Biotin (manufactured by Tokyo Chemical | 5 |
| Industry Co., Ltd.) | |
| DIEA | 10 |

<Cleavage of Peptide Attached with Spacer Sequence from Resin>

The resin binding to the peptide added with a spacer sequence was washed with diethyl ether (manufactured by FUJIFILM Wako Pure Chemical Corporation) and dried. This resin was brought into contact with a mixture solution of TFA:TIS:H₂O=95:2.5:2.5 at room temperature for 1 hour to cleave the peptide from the resin. The resin was removed from the solution by filtration, and 5 times the amount of ice-cooled diethyl ether was added to the solution, followed by inversion and stirring to generate a precipitate. The generated precipitate was centrifuged at 13,000 rpm for 3 minutes at 20° C., and the precipitate was again washed with diethyl ether and centrifuged under the same conditions. The precipitate was dried in a draft, and the resulting powder was stored at 4° C.

### <Evaluation of Interaction by ELISA>

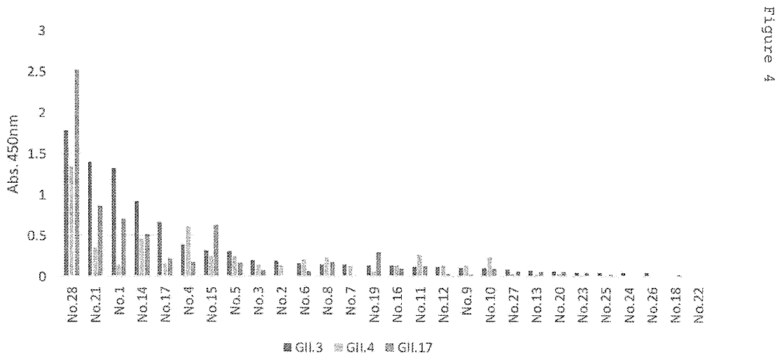

The peptide was dispersed in a 10% DMF aqueous solution. The concentration was calculated by an absorptiometer and was adjusted to 50 μM with a 10% DMF aqueous solution. This was added to Pierce™ Streptavidin Coated Plates, Clear, 96-Well (manufactured by Thermo Fisher Scientific) at 100 μL/well and was left to stand at room temperature for 1 hour. The supernatant was removed, and after washing with 200 μL of PBS-T (PBS containing 0.05% Tween 20) three times, 100 μL of a GII.3, GII.4, or GII.17 VLP solution diluted to 100 ng/mL with PBS-T was added, followed by leaving to stand at room temperature for 50 minutes. The supernatant was removed, and after washing with 200 μL of PBS-T three times, 100 μL of a rabbit anti-norovirus VLP polyclonal antibody (produced using a mixture of the GII.3 and GII.17 VLPs as an antigen by outsourcing to Eurofins Genomics K.K.) diluted to 1 μg/mL with a blocking agent was added, followed by leaving to stand at room temperature for 50 minutes. The supernatant was removed, and after washing with 200 μL of PBS-T three times, 100 μL of an HRP-labeled anti-rabbit IgG antibody (manufactured by Cell Signaling Technology, Inc.) diluted 1,000-fold with a blocking solution was added, followed by leaving to stand at room temperature for 50 minutes. The supernatant was removed, and after washing with 200 μL of PBS-T three times, 100 μL of 3,3′,5,5′-tetramethylbenzidine (manufactured by Abcam plc.) was added, followed by leaving to stand at room temperature for 15 minutes. As a reaction stopping solution, 100 μL of 0.5 M sulfuric acid was added, and the absorbance at 450 nm was measured with a multiplate reader (manufactured by Molecular Devices, LLC). The results are shown in FIG. 4.

### <Calculation of KD Value by Bio-Layer Interferometry: BLI Method>

The apparatus used was BLItz™ (manufactured by ForteBio). The tip of SA chip (manufactured by ForteBio) was kept in contact with purified water for 1 minute for hydration and was then kept in contact with a 1% BSA aqueous solution for 1 hour for blocking. The SA chip subjected to blocking treatment was set to the measuring unit of the BLItz™ main body, and measurement was performed according to the program shown in Table 14. The peptides of SEQ ID NOs: 1, 21, and 28 were used, and the concentration was adjusted to 100 μM. The concentration of the GII.3 VLP was adjusted to 1 and 0.1 mg/mL. The measurement data were analyzed using the attached software, and the KD value was calculated based on the Ka and Kd values. The results are shown in Table 15.

TABLE 14

| Operation | Used solution | Time [sec] |
|---|---|---|
| Baseline | PBS | 30 |
| Peptide bonding | 100 μM Peptide | 60 |
| Wash | PBS | 30 |
| Association | VLP | 90 |
| Dissociation | PBS | 60 |

TABLE 15

| | KD[M] | ka[1/Ms] | kd[1/s] |
|---|---|---|---|
| No. 1 | 6.01E−06 | 9.07E+03 | 5.46E−02 |
| No. 21 | 1.07E−04 | 3.58E+02 | 3.82E−02 |
| No. 28 | 2.18E−04 | 2.30E+02 | 5.00E−02 |

TABLE 16

| SEQ ID | | Appearance |
|---|---|---|
| NO: | Sequence | Frequency |
| 1 | GRRYYI | 23 |
| 2 | CLSNLA | 21 |
| 3 | GHRLHS | 21 |
| 4 | HLRSIR | 18 |
| 5 | RCSHLR | 18 |
| 6 | FLDTLG | 17 |
| 7 | GWHETE | 17 |
| 8 | PCSYFS | 17 |
| 9 | GEHAHS | 15 |
| 10 | GHHVSV | 15 |
| 11 | GHPNPR | 15 |
| 12 | LNWSRS | 15 |
| 13 | SVQSWK | 15 |
| 14 | RSVRMH | 14 |
| 15 | SIGVDR | 14 |
| 16 | LSKHSR | 13 |
| 17 | RIGHMR | 13 |
| 18 | SRWCLS | 13 |
| 19 | CTYVVE | 12 |
| 20 | DYVFCG | 12 |
| 21 | GKFFCH | 12 |
| 22 | GLRYRE | 12 |
| 23 | LKASIR | 12 |
| 24 | LRSGVN | 12 |
| 25 | NLYDRF | 12 |
| 26 | TWDLTL | 12 |
| 27 | VHYRQT | 12 |
| 28 | VMMQCP | 12 |
| 29 | DLLNSK | 11 |
| 30 | DRYSAW | 11 |
| 31 | HIPSRH | 11 |
| 32 | IMSSIG | 11 |
| 33 | SRHCVP | 11 |
| 34 | SVHTHR | 11 |
| 35 | TKRQNL | 11 |
| 36 | VNGVSH | 11 |
| 37 | WEVALH | 11 |
| 38 | AGYPRY | 10 |
| 39 | EMSRHC | 10 |
| 40 | FPFGST | 10 |
| 41 | GNIPGH | 10 |
| 42 | GNSPYT | 10 |
| 43 | HIHGRE | 10 |
| 44 | QSSKKF | 10 |
| 45 | RPFTML | 10 |
| 46 | RVSLYD | 10 |
| 47 | TTSHFK | 10 |
| 48 | VDSTSV | 10 |
| 49 | VETDGH | 10 |
| 50 | VIEMLD | 10 |
| 51 | VMCPNR | 10 |
| 52 | VRYPEI | 10 |
| 53 | ACRSAF | 9 |
| 54 | FDHFYS | 9 |
| 55 | GTRHPS | 9 |
| 56 | HRRGPS | 9 |
| 57 | HVRPFY | 9 |
| 58 | KFSHRK | 9 |
| 59 | MAWIGS | 9 |
| 60 | NTHRHS | 9 |
| 61 | PVRALC | 9 |
| 62 | RALAKR | 9 |
| 63 | REESFS | 9 |
| 64 | RKLFRN | 9 |
| 65 | SGTMQE | 9 |
| 66 | SGYYRV | 9 |
| 67 | SKHLAG | 9 |
| 68 | SSPRSH | 9 |
| 69 | VSVSLP | 9 |
| 70 | AVGCHV | 8 |
| 71 | CNLIAK | 8 |
| 72 | DFGQSS | 8 |
| 73 | EWAVSK | 8 |
| 74 | FPYARN | 8 |
| 75 | FTMHTN | 8 |
| 76 | GGSHST | 8 |
| 77 | GMLRFP | 8 |
| 78 | GQRLTV | 8 |
| 79 | GWYFHA | 8 |
| 80 | HICIRR | 8 |
| 81 | HRSNGH | 8 |
| 82 | ILGGHS | 8 |
| 83 | KTRAIN | 8 |
| 84 | LASSGH | 8 |
| 85 | LHMKPI | 8 |
| 86 | MSTHKV | 8 |
| 87 | SIESCL | 8 |
| 88 | SITVLY | 8 |
| 89 | SRSICS | 8 |
| 90 | SSCVMP | 8 |
| 91 | SSLALH | 8 |
| 92 | TGHIVW | 8 |
| 93 | THACAH | 8 |
| 94 | TVASNN | 8 |
| 95 | VGHVNW | 8 |
| 96 | VGRFPQ | 8 |
| 97 | VISLET | 8 |
| 98 | VVWFTD | 8 |
| 99 | VYIMNH | 8 |
| 100 | WDRPAF | 8 |
| 101 | YEGRND | 8 |
| 102 | AFTSHW | 7 |
| 103 | AHLHCD | 7 |
| 104 | DRVQSR | 7 |
| 105 | EEMIVS | 7 |
| 106 | FPTGTT | 7 |
| 107 | FRECLY | 7 |
| 108 | FWHARL | 7 |
| 109 | GCHYQF | 7 |
| 110 | GFAISR | 7 |
| 111 | GGGVTT | 7 |
| 112 | GHFDAR | 7 |
| 113 | GMSCGI | 7 |
| 114 | GSLDIM | 7 |
| 115 | GVNSGH | 7 |
| 116 | HHRLHG | 7 |
| 117 | HHYRGL | 7 |
| 118 | HSSFSS | 7 |
| 119 | HYRSKS | 7 |
| 120 | IGKGMV | 7 |
| 121 | ILISSC | 7 |
| 122 | ISRRGH | 7 |
| 123 | KRGGMV | 7 |
| 124 | KVHGST | 7 |
| 125 | LASMWR | 7 |
| 126 | LFHPWV | 7 |
| 127 | LISRHD | 7 |
| 128 | LWNMYD | 7 |
| 129 | MHSSLS | 7 |
| 130 | NASRFY | 7 |
| 131 | NHCPSR | 7 |
| 132 | NHDVRF | 7 |
| 133 | NKYSHR | 7 |
| 134 | NPKAHL | 7 |
| 135 | NRHLDP | 7 |
| 136 | PVLHLH | 7 |
| 137 | QDRVTS | 7 |
| 138 | QLHRHK | 7 |
| 139 | RFLMRH | 7 |
| 140 | RFVKRR | 7 |
| 141 | RHKVHH | 7 |
| 142 | RIVGSA | 7 |
| 143 | RRCKLL | 7 |
| 144 | RRGDNY | 7 |
| 145 | RRRTFH | 7 |
| 146 | RSAAGD | 7 |
| 147 | RSSSGR | 7 |
| 148 | RTGLLS | 7 |
| 149 | SDRVIA | 7 |
| 150 | STVRHR | 7 |
| 151 | STYRSW | 7 |
| 152 | SWPFCT | 7 |
| 153 | TTKYMW | 7 |
| 154 | TVLHHR | 7 |
| 155 | VMVGPS | 7 |
| 156 | VRNLVH | 7 |
| 157 | VRPSNG | 7 |
| 158 | VWYWTS | 7 |
| 159 | VYFIPD | 7 |
| 160 | WAGHRH | 7 |
| 161 | WVYARR | 7 |
| 162 | YRARMS | 7 |
| 163 | AFDGLG | 6 |
| 164 | AKQRFT | 6 |
| 165 | ARNHGS | 6 |
| 166 | ATITVW | 6 |
| 167 | CLHFAL | 6 |
| 168 | DKVCKN | 6 |
| 169 | DRWGSK | 6 |
| 170 | DSKLHN | 6 |
| 171 | DWPYSR | 6 |
| 172 | ECHHVH | 6 |
| 173 | EEKTRR | 6 |
| 174 | FGFREL | 6 |
| 175 | FPFYHR | 6 |
| 176 | FQYAHW | 6 |
| 177 | FTQDAV | 6 |
| 178 | GAKELS | 6 |
| 179 | GCLSHH | 6 |
| 180 | GFGQWI | 6 |
| 181 | GKVTFD | 6 |
| 182 | GLLIPF | 6 |
| 183 | GMDLRS | 6 |
| 184 | GPCNSA | 6 |
| 185 | GRNAGR | 6 |
| 186 | HDSRIR | 6 |
| 187 | HDYVSE | 6 |
| 188 | HFRRRV | 6 |
| 189 | HHNAPI | 6 |
| 190 | HKASHW | 6 |
| 191 | HLAHKK | 6 |
| 192 | HLGFHT | 6 |
| 193 | HMMSRL | 6 |
| 194 | HQTGRL | 6 |
| 195 | HSSLVT | 6 |
| 196 | HVGLDK | 6 |
| 197 | HWLRSV | 6 |
| 198 | HYRARY | 6 |
| 199 | IGRTFH | 6 |
| 200 | IHSPQK | 6 |
| 201 | IKAHTR | 6 |
| 202 | ILTHQH | 6 |
| 203 | ITLIDT | 6 |
| 204 | KACAHL | 6 |
| 205 | KHAWDL | 6 |
| 206 | KSMNNG | 6 |
| 207 | LHHSSY | 6 |
| 208 | LLGKPV | 6 |
| 209 | LNDMSF | 6 |
| 210 | LSLRSQ | 6 |
| 211 | LTALSV | 6 |
| 212 | MDHEMF | 6 |
| 213 | MQFELV | 6 |
| 214 | MSNKMV | 6 |
| 215 | NAVSLK | 6 |
| 216 | NCRTRP | 6 |
| 217 | NDHDHH | 6 |
| 218 | NGHSHA | 6 |
| 219 | NKSFHR | 6 |
| 220 | NPVHIK | 6 |
| 221 | NRVKPR | 6 |
| 222 | NSIAHR | 6 |
| 223 | NSRYFT | 6 |
| 224 | PDRFCA | 6 |
| 225 | PWTVPN | 6 |
| 226 | QCELKD | 6 |
| 227 | QFSQLG | 6 |
| 228 | QRVSFL | 6 |
| 229 | QVIQRL | 6 |
| 230 | QYCFVG | 6 |
| 231 | RGGRTL | 6 |
| 232 | RGHPST | 6 |
| 233 | RGTMWL | 6 |
| 234 | RHGQFF | 6 |
| 235 | RHTQTL | 6 |
| 236 | RIVLES | 6 |
| 237 | RKCIDR | 6 |
| 238 | RLHQCR | 6 |
| 239 | RLRDPQ | 6 |
| 240 | RNHSRI | 6 |
| 241 | RSSAKR | 6 |
| 242 | RTISGS | 6 |
| 243 | RTLLVQ | 6 |
| 244 | RTVPSS | 6 |
| 245 | RYMPVA | 6 |
| 246 | SAMFAS | 6 |
| 247 | SEHNRY | 6 |
| 248 | SHWRSY | 6 |
| 249 | SIITRR | 6 |
| 250 | SKVIRW | 6 |
| 251 | SKYTAL | 6 |
| 252 | SRSHWG | 6 |
| 253 | SSKSFA | 6 |
| 254 | STEFAR | 6 |
| 255 | STLVCP | 6 |
| 256 | STSVDW | 6 |
| 257 | TNRTVS | 6 |
| 258 | TNYLSF | 6 |
| 259 | VGMHHS | 6 |
| 260 | VMSPCN | 6 |
| 261 | VPFPHH | 6 |
| 262 | VPLLSR | 6 |
| 263 | VSDPVV | 6 |
| 264 | WTMSPA | 6 |
| 265 | WVFVKH | 6 |
| 266 | YGKQRG | 6 |
| 267 | YKASHG | 6 |
| 268 | YRSNHG | 6 |
| 269 | YYPDHP | 6 |

What is claimed is:

1. A norovirus-binding peptide consisting of an amino acid sequence selected from the group consisting of SEQ ID NOs: 1 to 269.

2. The norovirus-binding peptide according to claim 1, wherein the amino acid sequence selected from the group consisting of SEQ ID NOs: 1 to 269 is an amino acid sequence selected from the group consisting of SEQ ID NOs: 1, 4, 14, 15, 17, 21 and 28.

3. A norovirus-binding peptide consisting of the amino acid sequence selected from the group consisting of SEQ ID NOs: 1 to 269 according to claim 1 with a cysteine residue added to either or both of an N-terminus and a C-terminus thereof.

4. The norovirus-binding peptide according to claim 3, wherein the cysteine residue is added to the N-terminus and the C-terminus of the amino acid sequence selected from the group consisting of SEQ ID NOs: 1 to 269.

5. The norovirus-binding peptide according to claim 4, wherein the cysteine residue added to the N-terminus and the cysteine residue added to the C-terminus of the peptide consisting of an amino acid sequence selected from the group consisting of SEQ ID NOs: 1 to 269 are linked to each other via a disulfide bond to form a ring.

6. A norovirus-binding peptide consisting of an amino acid sequence of the peptide according to claim 1 with 1 to 20 amino acids added to either or both of the N-terminus and the C-terminus of the peptide.

7. A method for detecting norovirus comprising using the norovirus-binding peptide according to claim 1.

8. A norovirus detection kit comprising the norovirus-binding peptide according to claim 1.


SEQUENCE LISTING (292 sequences)

SEQ ID NO: 1 - protein, 6 aa, Artificial Sequence
  Designed peptide
GRRYYI

SEQ ID NO: 2 - protein, 6 aa, Artificial Sequence
  Designed peptide
CLSNLA

SEQ ID NO: 3 - protein, 6 aa, Artificial Sequence
  Designed peptide
GHRLHS

SEQ ID NO: 4 - protein, 6 aa, Artificial Sequence
  Designed peptide
HLRSIR

SEQ ID NO: 5 - protein, 6 aa, Artificial Sequence
  Designed peptide
RCSHLR

SEQ ID NO: 6 - protein, 6 aa, Artificial Sequence
  Designed peptide
FLDTLG

SEQ ID NO: 7 - protein, 6 aa, Artificial Sequence
  Designed peptide
GWHETE

SEQ ID NO: 8 - protein, 6 aa, Artificial Sequence
  Designed peptide
PCSYFS

SEQ ID NO: 9 - protein, 6 aa, Artificial Sequence
  Designed peptide
GEHAHS

SEQ ID NO: 10 - protein, 6 aa, Artificial Sequence
  Designed peptide
GHHVSV

SEQ ID NO: 11 - protein, 6 aa, Artificial Sequence
  Designed peptide
GHPNPR

SEQ ID NO: 12 - protein, 6 aa, Artificial Sequence
  Designed peptide
LNWSRS

SEQ ID NO: 13 - protein, 6 aa, Artificial Sequence
  Designed peptide
SVQSWK

SEQ ID NO: 14 - protein, 6 aa, Artificial Sequence
  Designed peptide
RSVRMH

SEQ ID NO: 15 - protein, 6 aa, Artificial Sequence
  Designed peptide
SIGVDR

SEQ ID NO: 16 - protein, 6 aa, Artificial Sequence
  Designed peptide
LSKHSR

SEQ ID NO: 17 - protein, 6 aa, Artificial Sequence
  Designed peptide
RIGHMR

SEQ ID NO: 18 - protein, 6 aa, Artificial Sequence
  Designed peptide
SRWCLS

SEQ ID NO: 19 - protein, 6 aa, Artificial Sequence
  Designed peptide
CTYVVE

SEQ ID NO: 20 - protein, 6 aa, Artificial Sequence
  Designed peptide
DYVFCG

SEQ ID NO: 21 - protein, 6 aa, Artificial Sequence
  Designed peptide
GKFFCH

SEQ ID NO: 22 - protein, 6 aa, Artificial Sequence
  Designed peptide
GLRYRE

SEQ ID NO: 23 - protein, 6 aa, Artificial Sequence
  Designed peptide
LKASIR

SEQ ID NO: 24 - protein, 6 aa, Artificial Sequence
  Designed peptide
LRSGVN

SEQ ID NO: 25 - protein, 6 aa, Artificial Sequence
  Designed peptide
NLYDRF

SEQ ID NO: 26 - protein, 6 aa, Artificial Sequence
  Designed peptide
TWDLTL

SEQ ID NO: 27 - protein, 6 aa, Artificial Sequence
  Designed peptide
VHYRQT

SEQ ID NO: 28 - protein, 6 aa, Artificial Sequence
  Designed peptide
VMMQCP

SEQ ID NO: 29 - protein, 6 aa, Artificial Sequence
  Designed peptide
DLLNSK

SEQ ID NO: 30 - protein, 6 aa, Artificial Sequence
  Designed peptide
DRYSAW

SEQ ID NO: 31 - protein, 6 aa, Artificial Sequence
  Designed peptide
HIPSRH

SEQ ID NO: 32 - protein, 6 aa, Artificial Sequence
  Designed peptide
IMSSIG

SEQ ID NO: 33 - protein, 6 aa, Artificial Sequence
  Designed peptide
SRHCVP

SEQ ID NO: 34 - protein, 6 aa, Artificial Sequence
  Designed peptide
SVHTHR

SEQ ID NO: 35 - protein, 6 aa, Artificial Sequence
  Designed peptide
TKRQNL

SEQ ID NO: 36 - protein, 6 aa, Artificial Sequence
  Designed peptide
VNGVSH

SEQ ID NO: 37 - protein, 6 aa, Artificial Sequence
  Designed peptide
WEVALH

SEQ ID NO: 38 - protein, 6 aa, Artificial Sequence
  Designed peptide
AGYPRY

SEQ ID NO: 39 - protein, 6 aa, Artificial Sequence
  Designed peptide
EMSRHC

SEQ ID NO: 40 - protein, 6 aa, Artificial Sequence
  Designed peptide
FPFGST

SEQ ID NO: 41 - protein, 6 aa, Artificial Sequence
  Designed peptide
GNIPGH

SEQ ID NO: 42 - protein, 6 aa, Artificial Sequence
  Designed peptide
GNSPYT

SEQ ID NO: 43 - protein, 6 aa, Artificial Sequence
  Designed peptide
HIHGRE

SEQ ID NO: 44 - protein, 6 aa, Artificial Sequence
  Designed peptide
QSSKKF

SEQ ID NO: 45 - protein, 6 aa, Artificial Sequence
  Designed peptide
RPFTML

SEQ ID NO: 46 - protein, 6 aa, Artificial Sequence
  Designed peptide
RVSLYD

SEQ ID NO: 47 - protein, 6 aa, Artificial Sequence
  Designed peptide
TTSHFK

SEQ ID NO: 48 - protein, 6 aa, Artificial Sequence
  Designed peptide
VDSTSV

SEQ ID NO: 49 - protein, 6 aa, Artificial Sequence
  Designed peptide
VETDGH

SEQ ID NO: 50 - protein, 6 aa, Artificial Sequence
  Designed peptide
VIEMLD

SEQ ID NO: 51 - protein, 6 aa, Artificial Sequence
  Designed peptide
VMCPNR

SEQ ID NO: 52 - protein, 6 aa, Artificial Sequence
  Designed peptide
VRYPEI

SEQ ID NO: 53 - protein, 6 aa, Artificial Sequence
  Designed peptide
ACRSAF

SEQ ID NO: 54 - protein, 6 aa, Artificial Sequence
  Designed peptide
FDHFYS

SEQ ID NO: 55 - protein, 6 aa, Artificial Sequence
  Designed peptide
GTRHPS

SEQ ID NO: 56 - protein, 6 aa, Artificial Sequence
  Designed peptide
HRRGPS

SEQ ID NO: 57 - protein, 6 aa, Artificial Sequence
  Designed peptide
HVRPFY

SEQ ID NO: 58 - protein, 6 aa, Artificial Sequence
  Designed peptide
KFSHRK

SEQ ID NO: 59 - protein, 6 aa, Artificial Sequence
  Designed peptide
MAWIGS

SEQ ID NO: 60 - protein, 6 aa, Artificial Sequence
  Designed peptide
NTHRHS

SEQ ID NO: 61 - protein, 6 aa, Artificial Sequence
  Designed peptide
PVRALC

SEQ ID NO: 62 - protein, 6 aa, Artificial Sequence
  Designed peptide
RALAKR

SEQ ID NO: 63 - protein, 6 aa, Artificial Sequence
  Designed peptide
REESFS

SEQ ID NO: 64 - protein, 6 aa, Artificial Sequence
  Designed peptide
RKLFRN

SEQ ID NO: 65 - protein, 6 aa, Artificial Sequence
  Designed peptide
SGTMQE

SEQ ID NO: 66 - protein, 6 aa, Artificial Sequence
  Designed peptide
SGYYRV

SEQ ID NO: 67 - protein, 6 aa, Artificial Sequence
  Designed peptide
SKHLAG

SEQ ID NO: 68 - protein, 6 aa, Artificial Sequence
  Designed peptide
SSPRSH

SEQ ID NO: 69 - protein, 6 aa, Artificial Sequence
  Designed peptide
VSVSLP

SEQ ID NO: 70 - protein, 6 aa, Artificial Sequence
  Designed peptide
AVGCHV

SEQ ID NO: 71 - protein, 6 aa, Artificial Sequence
  Designed peptide
CNLIAK

SEQ ID NO: 72 - protein, 6 aa, Artificial Sequence
  Designed peptide
DFGQSS

SEQ ID NO: 73 - protein, 6 aa, Artificial Sequence
  Designed peptide
EWAVSK

SEQ ID NO: 74 - protein, 6 aa, Artificial Sequence
  Designed peptide
FPYARN

SEQ ID NO: 75 - protein, 6 aa, Artificial Sequence
  Designed peptide
FTMHTN

SEQ ID NO: 76 - protein, 6 aa, Artificial Sequence
  Designed peptide
GGSHST

SEQ ID NO: 77 - protein, 6 aa, Artificial Sequence
  Designed peptide
GMLRFP

SEQ ID NO: 78 - protein, 6 aa, Artificial Sequence
  Designed peptide
GQRLTV

SEQ ID NO: 79 - protein, 6 aa, Artificial Sequence
  Designed peptide
GWYFHA

SEQ ID NO: 80 - protein, 6 aa, Artificial Sequence
  Designed peptide
HICIRR

SEQ ID NO: 81 - protein, 6 aa, Artificial Sequence
  Designed peptide
HRSNGH

SEQ ID NO: 82 - protein, 6 aa, Artificial Sequence
  Designed peptide
ILGGHS

SEQ ID NO: 83 - protein, 6 aa, Artificial Sequence
  Designed peptide
KTRAIN

SEQ ID NO: 84 - protein, 6 aa, Artificial Sequence
  Designed peptide
LASSGH

SEQ ID NO: 85 - protein, 6 aa, Artificial Sequence
  Designed peptide
LHMKPI

SEQ ID NO: 86 - protein, 6 aa, Artificial Sequence
  Designed peptide
MSTHKV

SEQ ID NO: 87 - protein, 6 aa, Artificial Sequence
  Designed peptide
SIESCL

SEQ ID NO: 88 - protein, 6 aa, Artificial Sequence
  Designed peptide
SITVLY

SEQ ID NO: 89 - protein, 6 aa, Artificial Sequence
  Designed peptide
SRSICS

SEQ ID NO: 90 - protein, 6 aa, Artificial Sequence
  Designed peptide
SSCVMP

SEQ ID NO: 91 - protein, 6 aa, Artificial Sequence
  Designed peptide
SSLALH

SEQ ID NO: 92 - protein, 6 aa, Artificial Sequence
  Designed peptide
TGHIVW

SEQ ID NO: 93 - protein, 6 aa, Artificial Sequence
  Designed peptide
THACAH

SEQ ID NO: 94 - protein, 6 aa, Artificial Sequence
  Designed peptide
TVASNN

SEQ ID NO: 95 - protein, 6 aa, Artificial Sequence
  Designed peptide
VGHVNW

SEQ ID NO: 96 - protein, 6 aa, Artificial Sequence
  Designed peptide
VGRFPQ

SEQ ID NO: 97 - protein, 6 aa, Artificial Sequence
  Designed peptide
VISLET

SEQ ID NO: 98 - protein, 6 aa, Artificial Sequence
  Designed peptide
VVWFTD

SEQ ID NO: 99 - protein, 6 aa, Artificial Sequence
  Designed peptide
VYIMNH

SEQ ID NO: 100 - protein, 6 aa, Artificial Sequence
  Designed peptide
WDRPAF

SEQ ID NO: 101 - protein, 6 aa, Artificial Sequence
  Designed peptide
YEGRND

SEQ ID NO: 102 - protein, 6 aa, Artificial Sequence
  Designed peptide
AFTSHW

SEQ ID NO: 103 - protein, 6 aa, Artificial Sequence
  Designed peptide
AHLHCD

SEQ ID NO: 104 - protein, 6 aa, Artificial Sequence
  Designed peptide
DRVQSR

SEQ ID NO: 105 - protein, 6 aa, Artificial Sequence
  Designed peptide
EEMIVS

SEQ ID NO: 106 - protein, 6 aa, Artificial Sequence
  Designed peptide
FPTGTT

SEQ ID NO: 107 - protein, 6 aa, Artificial Sequence
  Designed peptide
FRECLY

SEQ ID NO: 108 - protein, 6 aa, Artificial Sequence
  Designed peptide
FWHARL

SEQ ID NO: 109 - protein, 6 aa, Artificial Sequence
  Designed peptide
GCHYQF

SEQ ID NO: 110 - protein, 6 aa, Artificial Sequence
  Designed peptide
GFAISR

SEQ ID NO: 111 - protein, 6 aa, Artificial Sequence
  Designed peptide
GGGVTT

SEQ ID NO: 112 - protein, 6 aa, Artificial Sequence
  Designed peptide
GHFDAR

SEQ ID NO: 113 - protein, 6 aa, Artificial Sequence
  Designed peptide
GMSCGI

SEQ ID NO: 114 - protein, 6 aa, Artificial Sequence
  Designed peptide
GSLDIM

SEQ ID NO: 115 - protein, 6 aa, Artificial Sequence
  Designed peptide
GVNSGH

SEQ ID NO: 116 - protein, 6 aa, Artificial Sequence
  Designed peptide
HHRLHG

SEQ ID NO: 117 - protein, 6 aa, Artificial Sequence
  Designed peptide
HHYRGL

SEQ ID NO: 118 - protein, 6 aa, Artificial Sequence
  Designed peptide
HSSFSS

SEQ ID NO: 119 - protein, 6 aa, Artificial Sequence
  Designed peptide
HYRSKS

SEQ ID NO: 120 - protein, 6 aa, Artificial Sequence
  Designed peptide
IGKGMV

SEQ ID NO: 121 - protein, 6 aa, Artificial Sequence
  Designed peptide
ILISSC

SEQ ID NO: 122 - protein, 6 aa, Artificial Sequence
  Designed peptide
ISRRGH

SEQ ID NO: 123 - protein, 6 aa, Artificial Sequence
  Designed peptide
KRGGMV

SEQ ID NO: 124 - protein, 6 aa, Artificial Sequence
  Designed peptide
KVHGST

SEQ ID NO: 125 - protein, 6 aa, Artificial Sequence
  Designed peptide
LASMWR

SEQ ID NO: 126 - protein, 6 aa, Artificial Sequence
  Designed peptide
LFHPWV

SEQ ID NO: 127 - protein, 6 aa, Artificial Sequence
  Designed peptide
LISRHD

SEQ ID NO: 128 - protein, 6 aa, Artificial Sequence
  Designed peptide
LWNMYD

SEQ ID NO: 129 - protein, 6 aa, Artificial Sequence
  Designed peptide
MHSSLS

SEQ ID NO: 130 - protein, 6 aa, Artificial Sequence
  Designed peptide
NASRFY

SEQ ID NO: 131 - protein, 6 aa, Artificial Sequence
  Designed peptide
NHCPSR

SEQ ID NO: 132 - protein, 6 aa, Artificial Sequence
  Designed peptide
NHDVRF

SEQ ID NO: 133 - protein, 6 aa, Artificial Sequence
  Designed peptide
NKYSHR

SEQ ID NO: 134 - protein, 6 aa, Artificial Sequence
  Designed peptide
NPKAHL

SEQ ID NO: 135 - protein, 6 aa, Artificial Sequence
  Designed peptide
NRHLDP

SEQ ID NO: 136 - protein, 6 aa, Artificial Sequence
  Designed peptide
PVLHLH

SEQ ID NO: 137 - protein, 6 aa, Artificial Sequence
  Designed peptide
QDRVTS

SEQ ID NO: 138 - protein, 6 aa, Artificial Sequence
  Designed peptide
QLHRHK

SEQ ID NO: 139 - protein, 6 aa, Artificial Sequence
  Designed peptide
RFLMRH

SEQ ID NO: 140 - protein, 6 aa, Artificial Sequence
  Designed peptide
RFVKRR

SEQ ID NO: 141 - protein, 6 aa, Artificial Sequence
  Designed peptide
RHKVHH

SEQ ID NO: 142 - protein, 6 aa, Artificial Sequence
  Designed peptide
RIVGSA

SEQ ID NO: 143 - protein, 6 aa, Artificial Sequence
  Designed peptide
RRCKLL

SEQ ID NO: 144 - protein, 6 aa, Artificial Sequence
  Designed peptide
RRGDNY

SEQ ID NO: 145 - protein, 6 aa, Artificial Sequence
  Designed peptide
RRRTFH

SEQ ID NO: 146 - protein, 6 aa, Artificial Sequence
  Designed peptide
RSAAGD

SEQ ID NO: 147 - protein, 6 aa, Artificial Sequence
  Designed peptide
RSSSGR

SEQ ID NO: 148 - protein, 6 aa, Artificial Sequence
  Designed peptide
RTGLLS

SEQ ID NO: 149 - protein, 6 aa, Artificial Sequence
  Designed peptide
SDRVIA

SEQ ID NO: 150 - protein, 6 aa, Artificial Sequence
  Designed peptide
STVRHR

SEQ ID NO: 151 - protein, 6 aa, Artificial Sequence
  Designed peptide
STYRSW

SEQ ID NO: 152 - protein, 6 aa, Artificial Sequence
  Designed peptide
SWPFCT

SEQ ID NO: 153 - protein, 6 aa, Artificial Sequence
  Designed peptide
TTKYMW

SEQ ID NO: 154 - protein, 6 aa, Artificial Sequence
  Designed peptide
TVLHHR

SEQ ID NO: 155 - protein, 6 aa, Artificial Sequence
  Designed peptide
VMVGPS

SEQ ID NO: 156 - protein, 6 aa, Artificial Sequence
  Designed peptide
VRNLVH

SEQ ID NO: 157 - protein, 6 aa, Artificial Sequence
  Designed peptide
VRPSNG

SEQ ID NO: 158 - protein, 6 aa, Artificial Sequence
  Designed peptide
VWYWTS

SEQ ID NO: 159 - protein, 6 aa, Artificial Sequence
  Designed peptide
VYFIPD

SEQ ID NO: 160 - protein, 6 aa, Artificial Sequence
  Designed peptide
WAGHRH

SEQ ID NO: 161 - protein, 6 aa, Artificial Sequence
  Designed peptide
WVYARR

SEQ ID NO: 162 - protein, 6 aa, Artificial Sequence
  Designed peptide
YRARMS

SEQ ID NO: 163 - protein, 6 aa, Artificial Sequence
  Designed peptide
AFDGLG

SEQ ID NO: 164 - protein, 6 aa, Artificial Sequence
  Designed peptide
AKQRFT

SEQ ID NO: 165 - protein, 6 aa, Artificial Sequence
  Designed peptide
ARNHGS

SEQ ID NO: 166 - protein, 6 aa, Artificial Sequence
  Designed peptide
ATITVW

SEQ ID NO: 167 - protein, 6 aa, Artificial Sequence
  Designed peptide
CLHFAL

SEQ ID NO: 168 - protein, 6 aa, Artificial Sequence
  Designed peptide
DKVCKN

SEQ ID NO: 169 - protein, 6 aa, Artificial Sequence
  Designed peptide
DRWGSK

SEQ ID NO: 170 - protein, 6 aa, Artificial Sequence
  Designed peptide
DSKLHN

SEQ ID NO: 171 - protein, 6 aa, Artificial Sequence
  Designed peptide
DWPYSR

SEQ ID NO: 172 - protein, 6 aa, Artificial Sequence
  Designed peptide
ECHHVH

SEQ ID NO: 173 - protein, 6 aa, Artificial Sequence
  Designed peptide
EEKTRR

SEQ ID NO: 174 - protein, 6 aa, Artificial Sequence
  Designed peptide
FGFREL

SEQ ID NO: 175 - protein, 6 aa, Artificial Sequence
  Designed peptide
FPFYHR

SEQ ID NO: 176 - protein, 6 aa, Artificial Sequence
  Designed peptide
FQYAHW

SEQ ID NO: 177 - protein, 6 aa, Artificial Sequence
  Designed peptide
FTQDAV

SEQ ID NO: 178 - protein, 6 aa, Artificial Sequence
  Designed peptide
GAKELS

SEQ ID NO: 179 - protein, 6 aa, Artificial Sequence
  Designed peptide
GCLSHH

SEQ ID NO: 180 - protein, 6 aa, Artificial Sequence
  Designed peptide
GFGQWI

SEQ ID NO: 181 - protein, 6 aa, Artificial Sequence
  Designed peptide
GKVTFD

SEQ ID NO: 182 - protein, 6 aa, Artificial Sequence
  Designed peptide
GLLIPF

SEQ ID NO: 183 - protein, 6 aa, Artificial Sequence
  Designed peptide
GMDLRS

SEQ ID NO: 184 - protein, 6 aa, Artificial Sequence
  Designed peptide
GPCNSA

SEQ ID NO: 185 - protein, 6 aa, Artificial Sequence
  Designed peptide
GRNAGR

SEQ ID NO: 186 - protein, 6 aa, Artificial Sequence
  Designed peptide
HDSRIR

SEQ ID NO: 187 - protein, 6 aa, Artificial Sequence
  Designed peptide
HDYVSE

SEQ ID NO: 188 - protein, 6 aa, Artificial Sequence
  Designed peptide
HFRRRV

SEQ ID NO: 189 - protein, 6 aa, Artificial Sequence
  Designed peptide
HHNAPI

SEQ ID NO: 190 - protein, 6 aa, Artificial Sequence
  Designed peptide
HKASHW

SEQ ID NO: 191 - protein, 6 aa, Artificial Sequence
  Designed peptide
HLAHKK

SEQ ID NO: 192 - protein, 6 aa, Artificial Sequence
  Designed peptide
HLGFHT

SEQ ID NO: 193 - protein, 6 aa, Artificial Sequence
  Designed peptide
HMMSRL

SEQ ID NO: 194 - protein, 6 aa, Artificial Sequence
  Designed peptide
HQTGRL

SEQ ID NO: 195 - protein, 6 aa, Artificial Sequence
  Designed peptide
HSSLVT

SEQ ID NO: 196 - protein, 6 aa, Artificial Sequence
  Designed peptide
HVGLDK

SEQ ID NO: 197 - protein, 6 aa, Artificial Sequence
  Designed peptide
HWLRSV

SEQ ID NO: 198 - protein, 6 aa, Artificial Sequence
  Designed peptide
HYRARY

SEQ ID NO: 199 - protein, 6 aa, Artificial Sequence
  Designed peptide
IGRTFH

SEQ ID NO: 200 - protein, 6 aa, Artificial Sequence
  Designed peptide
IHSPQK

SEQ ID NO: 201 - protein, 6 aa, Artificial Sequence
  Designed peptide
IKAHTR

SEQ ID NO: 202 - protein, 6 aa, Artificial Sequence
  Designed peptide
ILTHQH

SEQ ID NO: 203 - protein, 6 aa, Artificial Sequence
  Designed peptide
ITLIDT

SEQ ID NO: 204 - protein, 6 aa, Artificial Sequence
  Designed peptide
KACAHL

SEQ ID NO: 205 - protein, 6 aa, Artificial Sequence
  Designed peptide
KHAWDL

SEQ ID NO: 206 - protein, 6 aa, Artificial Sequence
  Designed peptide
KSMNNG

SEQ ID NO: 207 - protein, 6 aa, Artificial Sequence
  Designed peptide
LHHSSY

SEQ ID NO: 208 - protein, 6 aa, Artificial Sequence
  Designed peptide
LLGKPV

SEQ ID NO: 209 - protein, 6 aa, Artificial Sequence
  Designed peptide
LNDMSF

SEQ ID NO: 210 - protein, 6 aa, Artificial Sequence
  Designed peptide
LSLRSQ

SEQ ID NO: 211 - protein, 6 aa, Artificial Sequence
  Designed peptide
LTALSV

SEQ ID NO: 212 - protein, 6 aa, Artificial Sequence
  Designed peptide
MDHEMF

SEQ ID NO: 213 - protein, 6 aa, Artificial Sequence
  Designed peptide
MQFELV

SEQ ID NO: 214 - protein, 6 aa, Artificial Sequence
  Designed peptide
MSNKMV

SEQ ID NO: 215 - protein, 6 aa, Artificial Sequence
  Designed peptide
NAVSLK

SEQ ID NO: 216 - protein, 6 aa, Artificial Sequence
  Designed peptide
NCRTRP

SEQ ID NO: 217 - protein, 6 aa, Artificial Sequence
  Designed peptide
NDHDHH

SEQ ID NO: 218 - protein, 6 aa, Artificial Sequence
  Designed peptide
NGHSHA

SEQ ID NO: 219 - protein, 6 aa, Artificial Sequence
  Designed peptide
NKSFHR

SEQ ID NO: 220 - protein, 6 aa, Artificial Sequence
  Designed peptide
NPVHIK

SEQ ID NO: 221 - protein, 6 aa, Artificial Sequence
  Designed peptide
NRVKPR

SEQ ID NO: 222 - protein, 6 aa, Artificial Sequence
  Designed peptide
NSIAHR

SEQ ID NO: 223 - protein, 6 aa, Artificial Sequence
  Designed peptide
NSRYFT

SEQ ID NO: 224 - protein, 6 aa, Artificial Sequence
  Designed peptide
PDRFCA

SEQ ID NO: 225 - protein, 6 aa, Artificial Sequence
  Designed peptide
PWTVPN

SEQ ID NO: 226 - protein, 6 aa, Artificial Sequence
  Designed peptide
QCELKD

SEQ ID NO: 227 - protein, 6 aa, Artificial Sequence
  Designed peptide
QFSQLG

SEQ ID NO: 228 - protein, 6 aa, Artificial Sequence
  Designed peptide
QRVSFL

SEQ ID NO: 229 - protein, 6 aa, Artificial Sequence
  Designed peptide
QVIQRL

SEQ ID NO: 230 - protein, 6 aa, Artificial Sequence
  Designed peptide
QYCFVG

SEQ ID NO: 231 - protein, 6 aa, Artificial Sequence
  Designed peptide
RGGRTL

SEQ ID NO: 232 - protein, 6 aa, Artificial Sequence
  Designed peptide
RGHPST

SEQ ID NO: 233 - protein, 6 aa, Artificial Sequence
  Designed peptide
RGTMWL

SEQ ID NO: 234 - protein, 6 aa, Artificial Sequence
  Designed peptide
RHGQFF

SEQ ID NO: 235 - protein, 6 aa, Artificial Sequence
  Designed peptide
RHTQTL

SEQ ID NO: 236 - protein, 6 aa, Artificial Sequence
  Designed peptide
RIVLES

SEQ ID NO: 237 - protein, 6 aa, Artificial Sequence
  Designed peptide
RKCIDR

SEQ ID NO: 238 - protein, 6 aa, Artificial Sequence
  Designed peptide
RLHQCR

SEQ ID NO: 239 - protein, 6 aa, Artificial Sequence
  Designed peptide
RLRDPQ

SEQ ID NO: 240 - protein, 6 aa, Artificial Sequence
  Designed peptide
RNHSRI

SEQ ID NO: 241 - protein, 6 aa, Artificial Sequence
  Designed peptide
RSSAKR

SEQ ID NO: 242 - protein, 6 aa, Artificial Sequence
  Designed peptide
RTISGS

SEQ ID NO: 243 - protein, 6 aa, Artificial Sequence
  Designed peptide
RTLLVQ

SEQ ID NO: 244 - protein, 6 aa, Artificial Sequence
  Designed peptide
RTVPSS

SEQ ID NO: 245 - protein, 6 aa, Artificial Sequence
  Designed peptide
RYMPVA

SEQ ID NO: 246 - protein, 6 aa, Artificial Sequence
  Designed peptide
SAMFAS

SEQ ID NO: 247 - protein, 6 aa, Artificial Sequence
  Designed peptide
SEHNRY

SEQ ID NO: 248 - protein, 6 aa, Artificial Sequence
  Designed peptide
SHWRSY

SEQ ID NO: 249 - protein, 6 aa, Artificial Sequence
  Designed peptide
SIITRR

SEQ ID NO: 250 - protein, 6 aa, Artificial Sequence
  Designed peptide
SKVIRW

SEQ ID NO: 251 - protein, 6 aa, Artificial Sequence
  Designed peptide
SKYTAL

SEQ ID NO: 252 - protein, 6 aa, Artificial Sequence
  Designed peptide
SRSHWG

SEQ ID NO: 253 - protein, 6 aa, Artificial Sequence
  Designed peptide
SSKSFA

SEQ ID NO: 254 - protein, 6 aa, Artificial Sequence
  Designed peptide
STEFAR

SEQ ID NO: 255 - protein, 6 aa, Artificial Sequence
  Designed peptide
STLVCP

SEQ ID NO: 256 - protein, 6 aa, Artificial Sequence
  Designed peptide
STSVDW

SEQ ID NO: 257 - protein, 6 aa, Artificial Sequence
  Designed peptide
TNRTVS

SEQ ID NO: 258 - protein, 6 aa, Artificial Sequence
  Designed peptide
TNYLSF

SEQ ID NO: 259 - protein, 6 aa, Artificial Sequence
  Designed peptide
VGMHHS

SEQ ID NO: 260 - protein, 6 aa, Artificial Sequence
  Designed peptide
VMSPCN

SEQ ID NO: 261 - protein, 6 aa, Artificial Sequence
  Designed peptide
VPFPHH

SEQ ID NO: 262 - protein, 6 aa, Artificial Sequence
  Designed peptide
VPLLSR

SEQ ID NO: 263 - protein, 6 aa, Artificial Sequence
  Designed peptide
VSDPVV

SEQ ID NO: 264 - protein, 6 aa, Artificial Sequence
  Designed peptide
WTMSPA

SEQ ID NO: 265 - protein, 6 aa, Artificial Sequence
  Designed peptide
WVFVKH

SEQ ID NO: 266 - protein, 6 aa, Artificial Sequence
  Designed peptide
YGKQRG

SEQ ID NO: 267 - protein, 6 aa, Artificial Sequence
  Designed peptide
YKASHG

SEQ ID NO: 268 - protein, 6 aa, Artificial Sequence
  Designed peptide
YRSNHG

SEQ ID NO: 269 - protein, 6 aa, Artificial Sequence
  Designed peptide
YYPDHP

SEQ ID NO: 270 - DNA, 50 bp, Artificial Sequence
  Synthetic oligonucleotide
cagacgtgtgctcttccgatctgatatcagatcggaagagcgtcgttaag

SEQ ID NO: 271 - DNA, 33 bp, Artificial Sequence
  Synthetic oligonucleotide
catcacaagtttgtacaaaaaagcaggctgtga

SEQ ID NO: 272 - DNA, 30 bp, Artificial Sequence
  Synthetic oligonucleotide
tatcaccactttgtacaagaaagctgggtt

SEQ ID NO: 273 - DNA, 31 bp, Artificial Sequence
  Synthetic oligonucleotide
atcacaagtttgtactgggagggcgatcgca

SEQ ID NO: 274 - DNA, 30 bp, Artificial Sequence
  Synthetic oligonucleotide
ctatcaccactttgttcgctacctcgcgaa

SEQ ID NO: 275 - DNA, 28 bp, Artificial Sequence
  Synthetic oligonucleotide
acaagtggtgatagcttgtcgagaagta

SEQ ID NO: 276 - DNA, 29 bp, Artificial Sequence
  Synthetic oligonucleotide
gtacaaacttgtgatgatccgcgcccgat

SEQ ID NO: 277 - DNA, 103 bp, Artificial Sequence
  Synthetic oligonucleotide
ccccaaataatgattttattttgactgatagtgacctgttcgttgcaacaaattgatgagcaatgcttttttataatgccaactttgtacaaaaaagcaggct

SEQ ID NO: 278 - DNA, 105 bp, Artificial Sequence
  Synthetic oligonucleotide
agcttacccagctttcttgtacaaagttggcattataagaaagcattgcttatcaatttgttgcaacgaacaggtcactatcagtcaaaataaaatcattatttg

SEQ ID NO: 279 - DNA, 217 bp, Artificial Sequence
  Synthetic oligonucleotide
  misc_feature (136)..(137): n is a, c, g, or t
  misc_feature (138)..(138): n is g, or t
  misc_feature (139)..(140): n is a, c, g, or t
  misc_feature (141)..(141): n is g, or t
  misc_feature (142)..(143): n is a, c, g, or t
  misc_feature (144)..(144): n is g, or t
  misc_feature (145)..(146): n is a, c, g, or t
  misc_feature (147)..(147): n is g, or t
  misc_feature (148)..(149): n is a, c, g, or t
  misc_feature (150)..(150): n is g, or t
  misc_feature (151)..(152): n is a, c, g, or t
  misc_feature (153)..(153): n is g, or t
gatcccgcgaaattaatacgactcactataggggaagtatttttacaacaattaccaacaacaacaacaaacaacaacaacattacattttacattctacaactacaagccaccatgggcagcggcggcagctgcnnnnnnnnnnnnnnnnnntgcgggggaggcagccatcatcatcatcatcacggcggaagcaggacggggggcggcgtggaaa

SEQ ID NO: 280 - DNA, 117 bp, Artificial Sequence
  Synthetic oligonucleotide
gatcccgcgaaattaatacgactcactataggggaagtatttttacaacaattaccaacaacaacaacaaacaacaacaacattacattttacattctacaactacaagccaccatg

SEQ ID NO: 281 - DNA, 78 bp, Artificial Sequence
  Synthetic oligonucleotide
  misc_feature (38)..(39): n is a, c, g, or t
  misc_feature (40)..(40): n is g, or t
  misc_feature (41)..(42): n is a, c, g, or t
  misc_feature (43)..(43): n is g, or t
  misc_feature (44)..(45): n is a, c, g, or t
  misc_feature (46)..(46): n is g, or t
  misc_feature (47)..(48): n is a, c, g, or t
  misc_feature (49)..(49): n is g, or t
  misc_feature (50)..(51): n is a, c, g, or t
  misc_feature (52)..(52): n is g, or t
  misc_feature (53)..(54): n is a, c, g, or t
  misc_feature (55)..(55): n is g, or t
acaactacaagccaccatgggcagcggcggcagctgcnnnnnnnnnnnnnnnnnntgcgggggaggcagccatcatca

SEQ ID NO: 282 - DNA, 61 bp, Artificial Sequence
  Synthetic oligonucleotide
tttccacgccgccccccgtcctgcttccgccgtgatgatgatgatgatggctgcctccccc

SEQ ID NO: 283 - DNA, 26 bp, Artificial Sequence
  Synthetic oligonucleotide
  misc_feature (1)..(1): Biotin-TEG-dA
  misc_feature (3)..(3): riboguanine
  misc_feature (12)..(12): 3-Cyanovinylcarbazole-dG
  misc_feature (23)..(23): Amino C6-dT
nanaatttccanccgccccccgncct

SEQ ID NO: 284 - DNA, 60 bp, Artificial Sequence
  Synthetic oligonucleotide
gatcccgcgaaattaatacgactcactataggggaagtatttttacaacaattaccaaca

SEQ ID NO: 285 - DNA, 22 bp, Artificial Sequence
  Synthetic oligonucleotide
tttccacgccgccccccgtcct

SEQ ID NO: 286 - DNA, 58 bp, Artificial Sequence
  Synthetic oligonucleotide
tcgtcggcagcgtcagatgtgtataagagacagcattctacaactacaagccaccatg

SEQ ID NO: 287 - DNA, 61 bp, Artificial Sequence
  Synthetic oligonucleotide
gtctcgtgggctcggagatgtgtataagagacagtttccacgccgccccccgtcctgcttc

SEQ ID NO: 288 - DNA, 47 bp, Artificial Sequence
  Synthetic oligonucleotide
caagcagaagacggcatacgagatactgagcggtctcgtgggctcgg

SEQ ID NO: 289 - DNA, 47 bp, Artificial Sequence
  Synthetic oligonucleotide
caagcagaagacggcatacgagattgcagctagtctcgtgggctcgg

SEQ ID NO: 290 - DNA, 51 bp, Artificial Sequence
  Synthetic oligonucleotide
aatgatacggcgaccaccgagatctacactctctccgtcgtcggcagcgtc

SEQ ID NO: 291 - DNA, 51 bp, Artificial Sequence
  Synthetic oligonucleotide
aatgatacggcgaccaccgagatctacaccgtctaattcgtcggcagcgtc

SEQ ID NO: 292 - DNA, 40 bp, Artificial Sequence
  Synthetic oligonucleotide
  misc_feature (1)..(1): Biotin-TEG-dA
  misc_feature (21)..(21): deoxyinosine
  misc_feature (27)..(27): 3-Cyanovinylcarbazole-dG
  misc_feature (38)..(38): Amino C6-dT
naaaaaaaaaaaaaaaaaaanttccanccgccccccgnct